SEQUENCE LISTING (32 sequences)

SEQ ID NO: 1 - protein, 183 aa, Escherichia coli
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGEMGKMVVTILSAVAQAERQRILERTNEGRQEAMAKGVVFGRKRKIDRDAVLNMWQQGLGASHISKTMNIARSTVYKVINESN

SEQ ID NO: 2 - protein, 185 aa, Escherichia coli
MRIFGYARVSTSQQSLDIQIRALKDAGVKANRIFTDKASGSSTDREGLDLLRMKVEEGDVILVKKLDRLGRDTADMIQLIKEFDAQGVAVRFIDDGISTDGDMGQMVVTILSAVAQAERRRILERTNEGRQEAKLKGIKFGRRRTVDRNVVLTLHQKGTGATEIAHQLSIARSTVYKILEDERAS

SEQ ID NO: 3 - protein, 186 aa, Escherichia coli
MTGQRIGYIRVSTFDQNPERQLEGVKVDRAFSDKASGKDVKRPQLEALISFARTGDTVVVHSMDRLARNLDDLRRIVQTLTQRGVHIEFVKEHLSFTGEDSPMANLMLSVMGAFAEFERALIRERQREGIALAKQRGAYRGRKKSLSSERIAELRQRVEAGEQKTKLAREFGISRETLYQYLRTDQ

SEQ ID NO: 4 - protein, 205 aa, Streptococcus pyogenes
MAKIGYARVSSKEQNLDRQLQALQGVSKVFSDKLSGQSVERPQLQAMLNYIREGDIVVVTELDRLGRNNKELTELMNAIQQKGATLEVLNLPSMNGIEDENLRRLINNLVIELYKYQAESERKRIKERQAQGIEIAKSKGKFKGRQHKFKENDPRLKHAFDLFLNGCSDKEVEEQTGINRRTFRRYRTRYNVTVDQRKNKGKRDS

SEQ ID NO: 5 - protein, 202 aa, Staphylococcus aureus
MIIGYARVSSLDQNLERQLENLKTFGAEKIFTEKQSGKSIENRPILQKALNFVRMGDRFIVESIDRLGRNYNEVIHTVNYLKDKEVQLMITSLPMMNEVIGNPLLDKFMKDLIIQILAMVSEQERNESKRRQAQGIQVAKEKGVYKGRPLLYSPNAKDPQKRVIYHRVVEMLEEGQAISKIAKEVNITRQTVYRIKHDNGLS

SEQ ID NO: 6 - protein, 201 aa, Xanthomonas campestris
MKIGYARVSTREQNPALQVDSLKAAGCERIYQDVASGAKTARPALDELLGQLRGGDVLVIWKLDRMGRSLKHLVELVGSLMERKVGLLSLNDPIDTTSAQGRFVFNLFATLAEFERELIRERTQAGLTAARARGRVGGRPKGLSPQAEATALAAETLYRERKLSVAAIAQKLHLSKSTLYSYLRHRGVEIGPYKQSAQSPI

SEQ ID NO: 7 - protein, 193 aa, Enterobacteria phage Mu
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSAMGRFFFHVMGALAEMERELIIERTMAGLAAARNKGRIGGRPPKLTKAEWEQAGRLLAQGIPRKQVALIYDVALSTLYKKHPAKRAHIENDDRIN

SEQ ID NO: 8 - protein, 190 aa, Salmonella typhimurium
MATIGYIRVSTIDQNIDLQRNALTSANCDRIFEDRISGKIANRPGLKRALKYVNKGDTLVVWKLDRLGRSVKNLVALISELHERGAHFHSLTDSIDTSSAMGRFFFHVMSALAEMERELIVERTLAGLAAARAQGRLGGRPRAINKHEQEQISRLLEKGHPRQQLAIIFGIGVSTLYRYFPASSIKKRMN

SEQ ID NO: 9 - protein, 213 aa, Methanococcus jannaschii
MMIMERHYTLKEASKILGVSIKTLQRWDKAGKIKCIRTLGGKRRVPESEIKRILGIKDKEQRKIIGYARVSFNAQKDDLERQIQLIKSYAEENGWDIQILKDIGSGLNEKRKNYKKLLKMVMNRKVEKVIIAYPDRLTRFGFETLKEFFKSYGTEIVIINKKHKTPQEELVEDLITIVSHFAGKLYGMHSHKYKKLTKTVKEIVREEDAKEKE

SEQ ID NO: 10 - protein, 217 aa, Helicobacter pylori
MNKRMLSIGQASKLLGVTIQTLRNWDKKDLLKPDELTKGGERRYKLESLRRINRSIVFNQDELKTIAYARVSSHDQQDDLIRQVQVLELYCARCGFNYEVIQDLGSGMNYYKKGLTKLLNLILDNQVKRLVLTHKDRLLRFGAELVFSICEAKGVEVVIINKGDENVRFEEELAKDVLEIITVFSARLYGSRSKKNKKLLDEMQEVITNNVSYLNHA

SEQ ID NO: 11 - protein, 159 aa, Staphylococcus aureus
MKQAIGYLRQSTTKQQSLAAQKQTIEALAKKHNIQYITFYSDKQSGRTDKRNGYQQITELIQQGQCDVLCCYRLNRLHRNLKNALKLMKLCQKYHVHILSVHDGYFDMDKAFDRLKLNIFISLAELESDNIGEQVKNGIKEKAKQGKMITTHAPFGYHY

SEQ ID NO: 12 - protein, 169 aa, Clostridium perfringens
MSRTSRITALYERLSRDDDLTGESNSITNQKKYLEDYARRNGFENIRHFTDDGFSGVNFNRPGFQSLIKEVEAGNVETLIVKDMSRLGRNYLQVGFYTEVLFPQKNVRFLAINNSIDSNNASDNDFAPFLNIMNEWYAKDTSNKIKAIFDARMKDGKRCSGSIPYGYNR

SEQ ID NO: 13 - protein, 166 aa, Lactococcus lactis bacteriophage TP901-1
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYH

SEQ ID NO: 14 - protein, 185 aa, Bacteriophage phi-C31
MTQGVVTGVDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFEL

SEQ ID NO: 15 - DNA, 28 bp, Artificial sequence
  Z-box site
cgttcgaaatattataaattatcagaca

SEQ ID NO: 16 - DNA, 28 bp, Artificial sequence
  Z-box site
tgtctgataatttataatatttcgaacg

SEQ ID NO: 17 - protein, 11 aa, Artificial sequence
  Linker sequence
TVDRSSDPTSQ

SEQ ID NO: 18 - protein, 6 aa, Artificial sequence
  Linker sequence
GSGGSG

SEQ ID NO: 19 - protein, 9 aa, Artificial sequence
  Linker sequence
GSGGSGGSG

SEQ ID NO: 20 - protein, 12 aa, Artificial sequence
  Linker sequence
GSGGSGGSGGSG

SEQ ID NO: 21 - protein, 7 aa, Artificial sequence
  Linker sequence
GGGSGGG

SEQ ID NO: 22 - protein, 12 aa, Artificial sequence
  Linker sequence
GGGSGGGGSGGG

SEQ ID NO: 23 - protein, 13 aa, Artificial sequence
  Linker sequence
TVDRSSDPTSQTS

SEQ ID NO: 24 - protein, 8 aa, Artificial sequence
  Linker sequence
GSGGSGTS

SEQ ID NO: 25 - protein, 11 aa, Artificial sequence
  Linker sequence
GSGGSGGSGTS

SEQ ID NO: 26 - protein, 14 aa, Artificial sequence
  Linker sequence
GSGGSGGSGGSGTS

SEQ ID NO: 27 - protein, 9 aa, Artificial sequence
  Linker sequence
GGGSGGGTS

SEQ ID NO: 28 - protein, 14 aa, Artificial sequence
  Linker sequence
GGGSGGGGSGGGTS

SEQ ID NO: 29 - protein, 12 aa, Artificial sequence
  Linker sequence
NRVAQQLAGKQS

SEQ ID NO: 30 - protein, 10 aa, Artificial sequence
  Linker sequence
SDYTQNNIHP

SEQ ID NO: 31 - protein, 6 aa, Artificial sequence
  Linker sequence
TVDRTS

SEQ ID NO: 32 - protein, 10 aa, Artificial sequence
  Linker sequence
SDYTQNNIHX

(19) United States
(12) Patent Application Publication
(10) Pub. No.: US 2006/0172373 A1
(43) Pub. Date: Aug. 3, 2006

(54) Mutant Recombinases

(75) Inventors: William Marshall Stark, Glasgow, Central Scotland (GB); Aram Akopian, Glasgow, Central Scotland (GB)

Correspondence Address: MORGAN LEWIS & BOCKIUS LLP, 1111 PENNSYLVANIA AVENUE NW, WASHINGTON, DC, 20004, US

(73) Assignee: The University Court of the University of Glasgow, Glasgow, Central Scotland (GB)

(21) Appl. No.: 10/529,059

(22) PCT Filed: Sep. 25, 2003
(86) PCT No.: PCT/GB03/04169

§ 371 Date: Dec. 14, 2005

(30) Foreign Application Priority Data
Sep. 25, 2002 (GB) 0222229.7

(51) Int. Cl. C07H 21/04; C12P 21/06; C12N 9/88

(52) U.S. Cl. 435/69.1; 435/232; 435/320.1; 435/325; 536/23.2

(57) ABSTRACT

The invention provides hyperactive mutant recombinases and hybrid mutant recombinases, and methods for their identification. Also provided are nucleic acids encoding hyperactive mutant recombinases and hybrid recombinases, as well as vectors and host cells. Host cells include eukaryotic cells capable of expressing said recombinases and carrying out site-specific recombination in the cell. The mutant recombinases may be used, for example, in biotechnology, gene therapy or transgenic applications.

### FIELD OF THE INVENTION

The present invention relates to hyperactive mutant recombinases including hybrid mutant recombinases, and methods for their identification. The present invention also relates to vectors comprising nucleic acid encoding said recombinases, as well as cells, especially eukaryotic cells capable of expressing said recombinases and carrying out site-specific recombination in the cell. Use of said recombinases in biotechnology and/or gene therapy/transgenic applications is also provided, as well as novel recombination systems in a cell such as a eukaryotic cell, especially a mammalian cell.

### BACKGROUND TO THE INVENTION

Site-specific recombination is extensively used for genetic manipulations in vivo, and is central to many proposed approaches to gene therapy (Kilby et al, 1993; Nagy, 2000). It is generally used to site-specifically introduce or excise a DNA fragment (for example an engineered cassette) into or from the genomic DNA, in a controlled way (for example, at a specific stage of development, or following deliberate induction of the. recombinase). Nearly all current applications of site-specific recombination in eukaryotes use the loxP-Cre system from bacteriophage P1 (see review by Nagy, 2000).

Cre is a good recombinase for these purposes because of its short DNA recombination site (.loxP; 34bp), its stability in vivo and the robustness of its activity even in chromatin-associated DNA. Use of these site-specific recombination systems in eukaryotes depends on the introduction of target DNA containing the appropriate DNA recognition/recombination sites into the organism. However, there is a great deal of current interest in modifying site-specific recombinases so as to recognise natural sequences in eukaryote (e.g. human) genomes (Santoro and Schultz, 2002; Scimenti et al., 2001; Buchholz and Stewart, 2001).

The bacterial transposon Tn3, a member of the large ‘serine recombinase’ family, encodes a site-specific recombination system comprising a 114 bp DNA site res, and a serine recombinase resolvase. res contains three binding sites for resolvase dimers. Recombination takes place within a ‘synapse’, consisting of the intertwined pair of resolvase-bound res sites that are to recombine. Strand exchange occurs at the centre of the two binding site Is, and is catalysed by the resolvase dimers bound at site I. However, wild-type resolvase is inactive on a substrate containing just two site Is; the presence of the ‘accessory’ resolvase-binding sites, II and III, hereinafter referred to as acc (Blake, 1995), in each res is essential for normal activity.

The acc sequences and the resolvase subunits bound to them play an essential part in the imposition of these selectivities (reviewed by Grindley, 2002). Regulatory DNA sequences like acc are prevalent in natural site-specific recombination systems. They may be adjacent to or distant from the site of crossing over, and may bind subunits of the recombinase (as acc does) and/or other proteins. Their functions are to ensure that recombination occurs only at the right times and places (reviewed by Nash, 1996).

The 20 kDA resolvases of the transposons Tn3 and γδ are very similar (147 of 185 residues are identical). X-ray crystallography has yielded high resolution structures of γδ resolvase, both on its own and in a complex with site I of res (Sanderson et al., 1990; Rice and Steitz, 1994; Yang and Steitz, 1995;). However, the structure of the synapse is still not well-defined, despite much analysis. To build a functional synapse, at least three types of resolvase-resolvase interaction are thought to be required, two of which are represented in crystal structures.

The 1,2 interaction (Hughes et al., 1990) forms the resolvase dimer that is present in solution and complexes of resolvase bound to parts of res; it is found in all of the three published crystal structures.

The role of the 2,3′ interaction between resolvase diners, seen in crystals of the γδ resolvase protein but not the DNA-resolvase co-crystal, is more elusive. Mutation of single residues at this interface eliminates recombination activity. The mutants are defective in cooperative binding to res, and in synapsis (Hughes et al., 1990; Murley and Grindley, 1998). The 2,3′ interaction is an essential feature of several proposed structures for the synapse (Rice and Steitz, 1994; Grindley, 1994; Yang and Steitz, 1995; Murley and Grindley, 1998; Sarkis et al., 2001; Rowland et al., 2002).

A third interaction, not observed in any of the crystal structures, has been proposed to be required in order to bring two 1,2 dimers together in an arrangement suitable for catalysis of strand exchange at site I (Rice and Steitz, 1994; Yang and Steitz, 1995). This third interaction may also have other “non-catalytic” roles in synapsis.

In the recent synapse model-of Sarkis et al. (2001), the protein core comprises three “DNA-out” tetramers, interacting with each other at 2,3′ surfaces. In published work (Schwikardi and Droge, 2000), γδ resolvase and a mutant of it have been shown to be active in mammalian cells, on full res sites and (very inefficiently) on the 28 bp site I of res. Another related recombinase, Gin, has been shown to be active in plant protoplasts (Maeser and Kahmann, 1991). Moreover earlier work by the present inventors describes mutants of Tn3 resolvase that act on 28 bp site I (inefficiently) in E. coli or in vitro. (Arnold et al., 1999). Some more recent work has been disclosed in Sarkis et al. 2001. Nevertheless, there has been no disclosure of potential or actual use of these mutants in other organisms, e.g. mammalian cells, or for genetic engineering purposes, and moreover it is desirable to develop better mutant recombinases than those hitherto described.

While the concept of directing an enzyme to a chosen DNA sequence by attaching a DNA-binding domain from an unrelated protein may not be new, there are several examples of enzymes that have been fused to the Zif268 DNA-binding domain or derivatives, in order to direct activity to a new site (for example Bibikova et al., 2002, and references cited therein). This has not been done until now for any site-specific recombinase, because it is not known how to do it. However, sequence recognition by site-specific recombinases has been altered by mutagenesis, or by swapping domains between related proteins. The tyrosine recombinases Cre and FLP, for example, have been extensively mutated to try to achieve new sequence recognition, with partial success (Buchholz and Stewart, 2001; Santoro and Schultz, 2002; and references cited therein). Cre/Flp hybrid proteins with unusual properties (but no recombination activity) have been created (Shaikh and Sadowski, 2000), and phage lambda integrase has also been ‘spliced’ with a closely related protein in order to alter sequence recognition (Nunes-Duby et al., 1994). However, for all the tyrosine recombinases, it is not obvious how the DNA-binding and catalytic functions of the protein could be separated, so changing recognition completely by attaching a heterologous DNA binding domain or similar is currently implausible.

For the serine recombinases, the crystal structure of γδ resolvase bound to site I DNA (Yang and Steitz, 1995) shows that the ‘DNA-binding’ and ‘catalytic’ domains are folded separately and do not make an intimate interaction. It was previously known that the C-terminal 45 residues of Tn3/γδ resolvase (141-185) are largely responsible for specific DNA recognition, and that residues 1-140 contain the known catalytic functions. However, there is no suggestion in the art that catalysis could be achieved without the natural C-terminal domain or some other similar domain.

It was unlikely that specific catalytic residues were in the C-terminal domain, because several hybrid recombinases were active. In these hybrids, the C-terminal domain was exchanged for that of another quite closely related serine recombinase. The junction was so as to conserve exactly the positions of residues that were homologous in the two parents. Examples of hybrids were between parts of Tn3 and Tn21 resolvases, or Tn3 and Tn552 resolvases, or Tn3 and γδ resolvases, or Gin and ISXc5 resolvase (Avila et al., 1990; Schneider et al., 2000). Nevertheless, all of these hybrids were active only on long DNA sequences (full res sites), not on a short sequence like site I, and that only small changes in sequence recognition were achieved.

It is an object of the present invention to obviate and/or mitigate at least one of the aforementioned disadvantages.

It is another object of the present invention to provide novel mutant recombinases which may find use in gene therapy and/or other biotechnological applications and/or develop uses of mutant recombinases not hitherto suggested.

### SUMMARY OF THE INVENTION

At its most general, the present invention provides materials and methods relating to mutant recombinases which are able to act in an improved fashion as compared to the wild-type recombinase specifically, the inventors have determined several key mutations that can be made to the catalytic domain of a serine recombinase which enable the enzyme to catalyse strand replacement at site I without accessory binding site II and III. Further, the inventors have determined that the catalytic domain of the mutated serine recombinase remains active even when linked to a heterologous DNA binding domain.

Thus, in a first aspect, the present provides a serine recombinase comprising a catalytic domain and a DNA binding domain wherein said catalytic domain is mutated at G101 or at a position corresponding to G101 of the Tn3 resolvase. Preferably, the mutation is G101S

The invention also provides a serine recombinase comprising a catalytic domain and a DNA binding domain wherein said catalytic domain is mutated at Q105 or at a position corresponding to Q105 of Tn3 resolvase. Preferably, the mutation is Q105L.

The invention also provides a serine recombinase comprising a catalytic domain and a DNA binding domain wherein said catalytic domain is mutated at D102 or at a position corresponding to D102 of Tn3 resolvase, and wherein the serine recombinase is not a D102Y E124Q mutant. The mutation preferably is selected from D102Y, D102I, D102F, D102T, D102V, D102W or D102A.

The serine recombinases may further comprising one or more additional mutations selected from the group L105Q, V107M, V107L, V107F, Q105L, A117V, R121K, E124Q, E124A, A89T, F92S, M103I or at positions corresponding to these mutations in Tn3 resolvase.

The serine recombinase may be a Tn3 resolvase, Sin recombinase, γδ resolvase, Tn 21 resolvase, β resolvase, ISXc5 resolvase, Gin resolvase, Hin resolvase, Methanococcus jannaschii.resolvase, IS607 resolvase, ccrA1 resolvase, TN4451 resolvase, TP901-1 resolvase and ΦC31 resolvase.

Muatations may be made by any of addition, substitution or deletion of one or more amino acids. Preferably, mutations are made by was to substitution.

The invention also provides a catalytic domain of a serine recombinase where the catalytic domain has been mutated in accordance with the present invention.

In a second aspect of the present invention, there is provided a nucleic acid molecule comprising nucleic acid sequence encoding a mutated serine recombinase in accordance with the present invention, and fragments and derivatives thereof. The nucleic acid sequence may form part of an expression vector for expressing the mutated serine recombinase. The expression vector or the nucleic acid may be within a host cell.

In a third aspect of the present invention, there is provided a hybrid recombinase comprising a catalytic domain from a serine recombinase connected by way of a linker to a heterologous DNA binding domain wherein said hybrid recombinase is capable of binding nucleic acid by way of said DNA binding domain and said catalysing recombination of said DNA. The hybrid recombinase is described in more detail below.

In a fourth aspect, the present invention provides a nucleic acid molecule comprising a nucleic acid sequence encoding a hybrid serine recombinase, and fragments and derivatives thereof. Also provided are nucleic acid sequences encoding a catalytic domain of a mutant recombinase, a heterologous DNA-binding domain, and a linker sequence, and fragments and derivatives thereof.

Nucleic acid encoding a mutant or mutant hybrid serine recombinase may be DNA or RNA. DNA may be, for example, cDNA, genomic DNA or a synthetic oligonucleotide. RNA may be, for example, mRNA.

A nucleic acid sequence encoding a catalytic domain of a hyperactive mutant recombinase as described herein is preferably at least 100, at least 200, at least 300, or at least 400 base pairs in length. Preferably, the nucleic acid is less than 500 or less than 550 base pairs in length. Nucleic acid sequences of the invention may be, in particular, 420, 423 or 432 base pairs in length.

A nucleic acid sequence encoding a linker sequence of a hybrid mutant recombinase as described herein is preferably at least 6, at least 10, at least 20, at least 30 or at least 40 base pairs in length. Preferably, the nucleic acid is 6, 9, 12, 15, 18, 21, 24, 27, 30, 33, 36, 39, 42 or 45 base pairs in length.

It will be appreciated by the skilled person that a given mutant recombinase may be encoded by different nucleic acid sequences, due to the degeneracy of the genetic code.

Also provided are vectors comprising nucleic acid sequences encloding hyperactive mutant recombinases and hybrid mutant recombinases as described herein. Host cells containing said nucleic acid sequences or vectors are also provided.

In a further aspect the present invention provides a method for identifying a hyperactive mutant serine recombinase capable of catalysing site-specific DNA recombination when bound to a recognition site comprising fewer nucleotides than necessary for achieving recombination with a corresponding wild-type serine recombinase, comprising the steps of

mutating said wild-type serine recombinase such that the mutant recombinase comprises one or more mutations, in a catalytic domain of the recombinase, with respect to the wild-type serine recombinase; and

detecting whether or not said mutant serine recombinase is capable of catalysing DNA recombination when bound to said recognition site comprising fewer nucleotides than necessary for achieving recombination with the corresponding wild-type serine recombinase.

In yet another aspect of the present invention, there is provided a method of recombining DNA contacting a first DNA sequence and a second DNA sequence with a serine recombinase or a hybrid recombinase according to the invention under suitable conditions for allowing a recombination of said first and second DNA sequences

The term hyperactive mutant recombinase is used to indicate that the mutant is capable of recombinase activity at smaller recognition sites than required by a wild-type recombinase.

Generally speaking, recombination is carried out such that two such recognition sites are brought into close proximity for site-specific recombination to occur. Site-specific recombination is understood to relate to genetic recombination occurring between two particular, but not necessarily homologous, short DNA sequences, as in the integration or excision of phage DNA from a bacterial chromosome or in transposition.

It is likely that more than one detection step from wild-type to preferred mutant may be required. That is, it may be necessary to first select mutants with a substrate comprising one wild-type recognition site and one site of reduced size and to the further mutagenise suitable mutants to get a preferred hyperactive mutant which shows recombination activity at two sites of reduced size. Conveniently the sites of reduced size comprise less than 50 nucleotides, typically less than 30 nucleotides.

The present invention describes in one embodiment recombinases derived from Tn3 resolvase, by combining mutations as indicated below, that efficiently recombine two sequences corresponding to the 28 bp binding site I of Tn3 res (or minor variants thereof). The D102Y E124Q mutant described in Arnold et al. 1999 has weak activity on a site I×site I substrate, in E. coli or in vitro; insufficient to be useful and is not therefore encompassed within the scope of the present invention.

Much more active mutants were created by combining mutations in the region close to D102. Additionally, all the most efficient versions are mutant at D102. The present inventors have tested all possible residues at position 102; the effects of the single mutants are, in decreasing order of hyperactivity, Y, I>F, T, V, W>A> all others. Mutation of G101 has also been observed to cause a big effect; specifically to serine (G101S). Mutation of Q105 also had an effect, particularly to serine (Q105S). Thus, mutants according to the present invention preferably comprise mutations at D102 and/or G101 and/or Q105S or corresponding residues from other serine recombinases. Mutations at other residues can also promote hyperactivity; these include (in approximate order of strength of effect) V107M, V107F also increased hyperactivity. Preferably the mutant enzymes have combinations of two or more of these mutations.

It has also been found that mutations of resolvase surface residues corresponding to a ‘2,3′ interface’ enhanced the activity of the mutants; see hereinafter. The mutations that have been tested were R2A and E56K, but mutation of several nearby residues (Hughes et al., 1990) might be similarly effective. Thus, preferably the mutants of the present invention also comprise at least one mutation that affects the 2,3 interface.

Whilst the present inventors have focussed their work on Tn3 resolvase, it will be appreciated that the scope of the present invention may easily be extended to other serine recombinases, due to the similarity between members of the family.

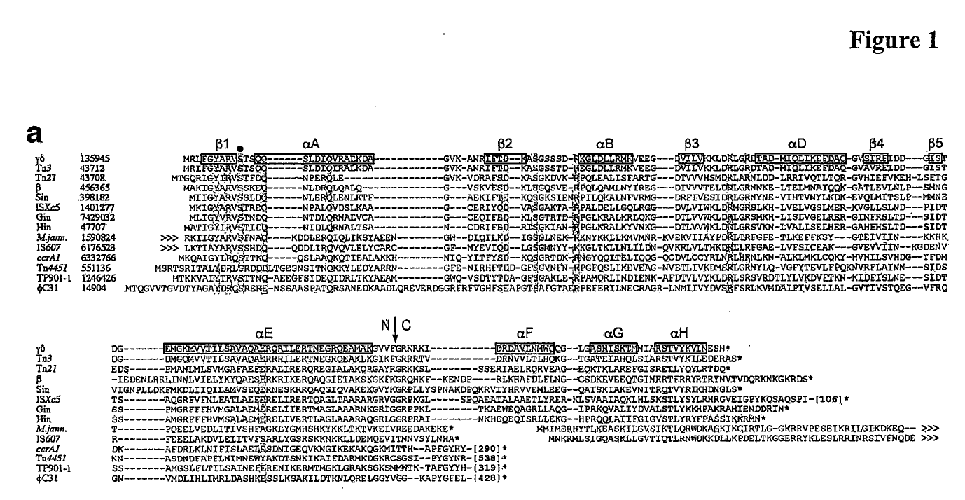

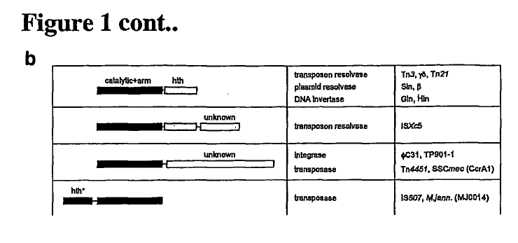

The serine recombinases comprise a large family of related enzymes, which can be identified by sequence homology using standard algorithms such as BLAST. Several residues are completely conserved, or nearly so, throughout the family. Structural features corresponding to particular parts of the primary sequence can be characterized because there are high-resolution crystal structures of the complete γδ resolvase protein, and a fragment of Hin, as well as a large body of other biochemical data that give information on the structures. Those skilled in the art can easily identify the residues in other serine recombinases that might correspond to the Tn3 residues which can be mutated to cause hyperactivity. For example, residues G101 and D102 are the two Tn3 resolvase residues immediately preceding the N-terminus of a long α-helix, the E-helix of Yang and Steitz 1995, that contributes to the dimer interface. The equivalent residues can be identified in most other members of the serine recombinase family. Similarly, residues corresponding to those involved in the 2,3′ interaction can be identified. See for example the review by Smith & Thorpe, 2002, or the attached alignment FIG. 1 which shows an alignment of a number of serine recombinases. For example, the present inventors have preliminary evidence that equivalent mutations of Sin recombinase from Staphylococcus aureus, which is quite distant from Tn3 resolvase, have the predicted effects.

The hyperactive mutants described herein can utilise the ‘Site I’ sequence for recombination. The ‘Site I’ is a 28 bp sequence from the natural res recombination site. Desirably smaller regions could be used which still cause recombination to occur. This may depend however on the mutant developed, but this can easily be determined by the skilled addressee. In practice, however, the sequence will always be embedded in a longer DNA molecule. It has been observed that many bases can be mutated individually without serious loss of recombination activity, and even multiple changes may not be very deleterious. However, a site comprising only the central 16 bp of site I (that is, 6 bp at each end replaced so that no bases are conserved), or <16 bp, is not a substrate for the hyperactive mutant resolvases described herein.

Advantages of Hyperactive Serine Recombinases Over Currently Available Enzymes for Genetic Manipulation

1. They act at short DNA sites, and do not require specific site orientation or supercoiling. They are therefore ‘better’ than other serine recombinases previously proposed for these uses. (Long sites and other requirements make it much more difficult to set up suitable constructs etc., and affect reactivity in chromatin-associated DNA).

2. They do not interact with tyrosine recombinases such as Cre or FLP, and act at different sites, so they can be used in applications where two (or more) independent recombination systems are required (see reviews etc.).

3. They may have advantages in real systems, because of their different properties and mechanism. For example, they might be more easily expressed/more stable in mammalian cells, or they might give more complete recombination.

In a further aspect, the present invention provides a hybrid mutant recombinase comprising an N-terminal catalytic domain from a serine recombinase connected by way of a linker region to a heterologous C-terminal DNA binding domain wherein the mutant recombinase is capable of binding nucleic acid by way of said DNA binding domain and said mutant recombinase catalysing recombination. Preferably the catalytic domain is from a hyperactive mutant recombinase identified, for example, according to the present invention.

It was previously known that the N-terminal domain of Tn3 resolvase (or any other serine recombinase tested) has no catalytic activity on its own, nor does the isolated N-terminal domain of mutants that act on site I. It was therefore surprising that attachment of an unrelated DNA-binding domain to a mutant catalytic domain could restore activity at a very different DNA site. Reasons why this might not have been considered feasible are:

1. The natural DNA-binding domain might play some essential part in the reaction mechanism, which could be performed by a related DNA-binding domain, but not an unrelated one; e.g. involvement of conserved residues, or transient dissociation from its binding site.

2. The natural domain might not participate in the reaction, but its size, shape, and position might be critical. For example, a larger domain might interfere with essential conformational changes in the DNA or protein.

3. The nature of the linker sequence between the two domains might be critical, and it might not have been possible to reconstruct it appropriately (for example, because the N-terminal residues of the unrelated DNA-binding domain and the resolvase DNA-binding domain were differently positioned relative to the binding site). In practice the important steps in going from a natural serine recombinase eg. Tn3 recombination system to a functional ‘hybrid’ system are as follows:

1. Identification of multiple mutants of resolvase that rapidly recombine two 28 bp site I's, thereby removing the requirement for ‘accessory sites’ (see Arnold et al., 1999; and the development of hyperactive mutants described herein);

2. Deciding where to terminate the N-terminal domain, to separate DNA-binding from essential catalytic functions;

3. Choosing of an appropriate substitute DNA-binding domain (e.g. Zif268) (from literature analysis);

4. Designing appropriate linker peptide sequences, to join the DNA-binding and catalytic domains of the hybrids; and

5. Designing of potential recombination sites for the hybrid enzyme.

In the hybrid recombinases of the present invention, the catalytic domain of a hyperactive mutant resolvase (or other serine recombinase) is joined via a short linker sequence to a DNA-binding domain from a different protein. The DNA-binding domain can be any of a number of such domains known to those skilled in the art, such as the domain from other serine recombinases, or from some transposases, or from bacterial repressors, tyrosine recombinases, etc. Suitably the DNA-binding domain may be eukaryotic in origin, for example, from eukaryotic transcription factors, especially a zinc finger DNA-binding domain such as that from Zif268, or variants of one of these with altered sequence recognition.

The hybrids that have been constructed to date by the present inventors contain the first 146 contiguous residues of Tn3 resolvase, with appropriate ‘activating’ mutations (see hereinabove for information). The proteins actually tested have all of the following mutations: R2A E56K G101S D102Y M1031 Q105L, although this should not be construed as limiting. The traditional ‘catalytic’ and DNA-binding, domains of resolvase and relatives were identified following proteolysis, and are residues 1-140 and 141-183 (for γδ resolvase) respectively. The C-terminal domain has been shown to retain DNA-binding activity, but no activities were found for the N-terminal ‘catalytic’ domain on its own. Current evidence suggests that all catalytic functions may reside in the contiguous residues 1-125. The sequence from 126-146 may however, contribute to binding and sequence recognition near the centre of the site. It is envisaged that it may be possible to mutate the 126-146 region or replace it with the equivalent segment from another serine recombinase, to alter reactivity or target specificity.

Preferably the linker region should be a sequence with structural flexibility, but the linker may depend strongly on the DNA-binding domain employed. This can however, easily be determined by the skilled addressee. It may be that shorter linkers will potentially lead to more efficient recombination, but might be more restricted in sequence variation. Thus an appropriate linker may depend on the requirements of the user. Some linkers may increase the efficiency of recombination at the expense of DNA sequence specificity, whilst others may allow recombination to occur at lower efficiency, but with a greater variation in sequence.

Resolvase binds to site I as a dimer. To act at asymmetric sequences (see below), it will be desirable to bind a heterodimer of the hybrid recombinase, where the DNA-binding domains of the two subunits interact with distinct sequence elements. Likewise, it may be desirable to have a different heterodimer to recognize a partner recombination site; i.e. up to four different hybrid recombinase proteins could be simultaneously involved, see Bibikova et al., 2002 for an example of this type of approach in a different system).

Although the hybrids of the present invention have been exemplified with respect to Tn3 resolvase-derived systems, this should not be construed as limiting. Based on the present teaching similar procedures could be used to create equivalent hybrids from other serine recombinases. Indeed, this might lead to better recombinases, because other recombinases have different ‘site I’ central sequences, which could be better for some specific natural sequences chosen to be recombination sites.

In order for a hybrid recombinase to function appropriately and catalyse recombination it is necessary for the enzyme to recognise and bind to an appropriate stretch of DNA. Typically the DNA sequence may comprise two regions recognized by the DNA-binding domain(s) of the hybrid recombinase(s), flanking a central sequence which may make some specific interactions with the catalytic domain and/or the 126-146 segment, or similar region from another serine recombinase. The site will always be embedded in a longer DNA molecule (typically, but not necessarily, kilobasepairs).

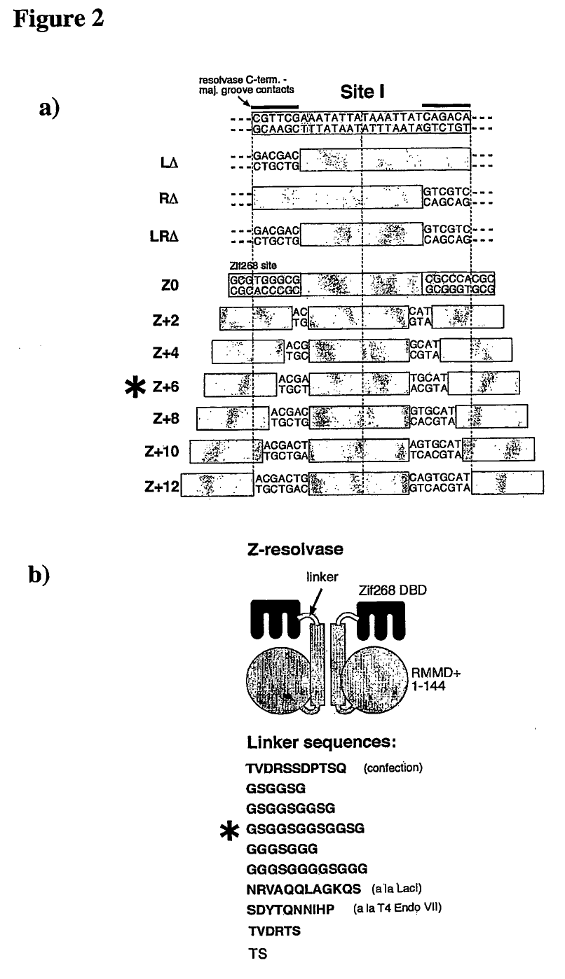

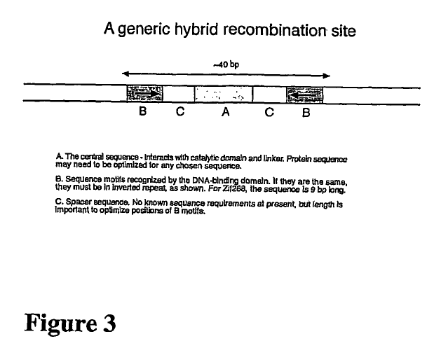

A typical site may be about 40 bp long. Experiments by the present inventors indicate that the positioning of the sequence elements that recognize the DNA-binding domains (relative to the centre of the site) is very important. The ideal positions will certainly vary depending on the DNA-binding domain and linker sequence used. The sequences of sites that have been tested by the present inventors are shown in attached FIG. 2a. These sites all comprise two copies of the natural 9 bp motif that is recognized by Zif268, flanking a central sequence of varying length. All of the central sequences used so far contain at least 11 contiguous basepairs of identity to the centre of site I, but it is very likely that sequences with less similarity to site I will also be active. It should be noted that non-hybrid hyperactive mutant resolvases are not active on these sites. The main features of the recombination site are illustrated in the attached FIG. 3.

The two sites that are to recombine need not be identical. They could be recognized by separate hybrid recombinase heterodimers, providing that the catalytic domains were similar, so that the catalytically competent synapse of the two sites could be formed. Importantly however, the 2 bp at the centre of the sites should be identical for efficient reaction (this is because these bp form a ‘heteroduplex’ in the recombinants, and the basepairs would be mismatched if the 2 bp sequences were different). (Tyrosine recombinases require longer regions of identity at the centre of their sites; 6 bp for Cre, and 8 bp for FLP). Also, the relative orientation of this ‘overlap’ sequence defines whether excision or inversion will occur between two sites in the same molecule.

Without being bound by theory it is predicted that, for any chosen recombination site sequence, it will be necessary to carry out an optimization procedure to achieve high activity. In general, this procedure will include the following steps.

1. One or two candidate recombination sites will be chosen, which have a central sequence with some similarity to site I, flanked by sequences at appropriate distances from the centre that could recognize selected DNA-binding domains;

2. The DNA-binding domains will be optimized for recognition of their targets. This can be done completely separately from the recombination system, using methods well known to those skilled in the art; mutagenesis followed by ‘phage display’ selection, swapping of parts from known variants of the DNA-binding domain, etc. (see reviews; e.g. Pabo et al., 2001);

3. Likewise, the catalytic domains and linkers may be optimized for interaction with and recombination at the central sequences. This may be done by making a trial recombination site, with the chosen central sequence placed between motifs recognized by a DNA-binding domain that is known to work well; for example, Zif268 itself. The catalytic domain and linker will then be optimized in essentially the same way as in (2), using mutagenesis/selection methods (e.g. as described in herein.), or splicing of parts from different variants or from different serine recombinases, etc; and

4. Complete candidate hybrid recombinases may then be assembled, and tested on the intact chosen sites. If necessary, efficiency of recombination at the sites may be improved by further rounds of mutagenesis and selection.

In a further aspect there is provided use of a hyperactive mutant recombinase, or hybrid recombinase according to the present invention for carrying out site-specific recombination.

Preferably site-specific recombination is carried out in a eukaryotic cell or on eukaryotic DNA. More preferably site-specific recombination is conducted in a mammalian cell or on mammalian DNA.

In a further aspect there is provided use of a hyperactive mutant recombinase, or hybrid recombinase according to the present invention for the manufacture of a medicament for therapy or prophylaxis. Said hyperactive mutant may be used to introduce a therapeutic gene or replace/remove a defective or deleterious gene sequence from the genome of a particular organism, such as a mammal.

In principle, all of the recombinases described herein could be used for virtually any current or envisaged applications of site-specific recombinases such as cell therapy, tissue engineering and/or gene therapy (see for example Gorman & Bullock, 2000 and references sited therein). The hybrid recombinases can also be used to create new sequence specificities in experimental systems, but more importantly, they can be used to target recombination to natural sequences in the genomes of (any) important organisms.

Advantage and Utility of Hybrid Recombinases

Potential applications are for example in WO 01/16345. Basically, a DNA segment containing useful (e.g. therapeutic) genes can be introduced at specific genomic sites, or ‘bad’ genes can be excised from the genomes of living cells, or control of gene function can be systematically altered by excision, integration, or inversion of DNA segments. Two examples are: (1) It may be possible to develop a potential therapy for HIV and other retroviral diseases, by excision of the proviral DNA (see below); (2) it may be possible to introduce useful genes (e.g. for antibodies) at specific sites in for example the casein gene loci of cows, so that they would express large quantities of the gene product in their milk.

Several groups are attempting to adapt the tyrosine recombinases Cre and FLP to recognize new sites (see, for example, Buchholz and Stewart, 2001; Santoro and Schultz, 2002). However, the present inventors believe that mutant serine recombinases are likely to be much more successful for this approach, because their modular structure facilitates the ‘hybrid’ constructions described herein. Also, they are likely to be much more suitable for recombining between two natural sites (e.g. for excision of natural genes), because they require only 2 bp of homology at the centre of the recombination sites for efficient reaction. Cre requires 6 bp and FLP requires 8 bp (Nash, 1996); pairs of sites with this degree of identity will be very rare.

Clearly the recombinase genes would need to be introduced into and expressed in the target cells, for most applications. Thus the present invention also provides vectors comprising a nucleic acid sequence encoding a hyperactive mutant recombinase or hybrid mutant recombinase as described herein.

The vector may be, for example, a plasmid vector. The vector may be an expression vector for expression of a protein or polypeptide from the nucleic acid sequence. The vector may contain a tag for purification of the protein or polypeptide, for example a His tag or a GST tag. The vector may also comprise one or more recombinase binding sites which are recognisable by said mutant recombinase. Said recognition site(s) may comprise a mutated sequence with respect to the native sequence recognisable by the unmutated recombinase.

Suitable vectors and methods for their production are well known in the art (see for example Sambrook & Russell, Molecular Cloning: A Laboratory Manual (3rd Edition), Cold Spring Harbor Laboratory Press 2001).

The present invention also provides a host cell containing a vector or isolated nucleic acid sequence as described above. Preferably, the host cell will permit expression of the mutant recombinase or hybrid recombinase from the vector or nucleic acid. The expressed protein may subsequently be released from the cell and purified for use in other applications. Alternatively, the expressed protein may serve as a recombinase within said cell.

It might be necessary to modify the recombinases for various reasons concerned with their properties in the target cells, such as to increase stability, direct them to the nucleus, allow their visualization. All such modifications are well known to those skilled in the art.

The present invention will now be further described by way of example and reference to the Figures which show:

FIG. 1 shows a sequence alignment of serine recombinase sequences.

a) An alignment of the sequences of selected serine recombinases (with accession numbers). The secondary structure elements of γδ resolvase, for which the crystal structure is known, are shown. An arrow marks the junction between the N— and C-terminal fragments of gamma delta resolvase obtained by proteolysis. Conserved residues in or near the active site are highlighted (shaded grey); S10 (Tn3/γδ numbering) is marked (o). The number of residues in a C-terminal extension to a sequence (not shown) is in brackets. The C-terminus is indicated by an asterisk. For the Methanococcus jannaschii (‘M.jann.’) and IS607 transposase sequences, the N— (blue) and C-terminal domains are aligned with the C— and N-terminal domains, respectively, of γδ resolvase. b) A cartoon showing the domain structures of the recombinases in (a).

FIG. 2a shows details of Z-box sites which have been tested by the present inventors.

FIG. 2b shows details of the flexible linkers which have been tested by the present inventors.

FIG. 3 shows a schematic representation of a generic hybrid recombination site.

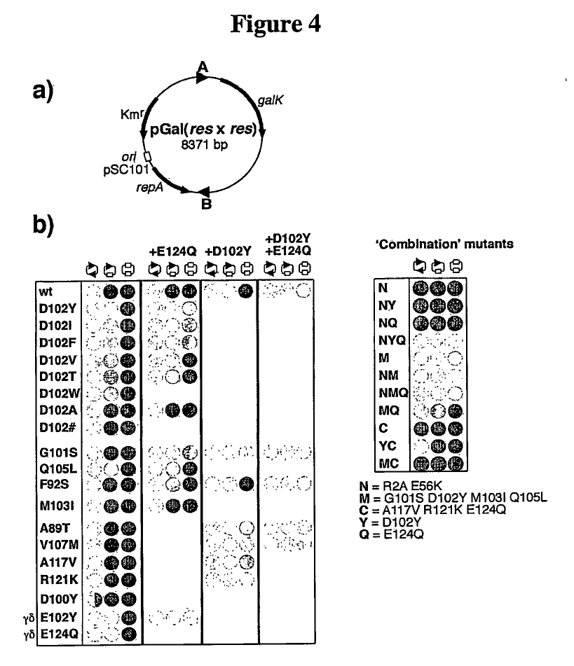

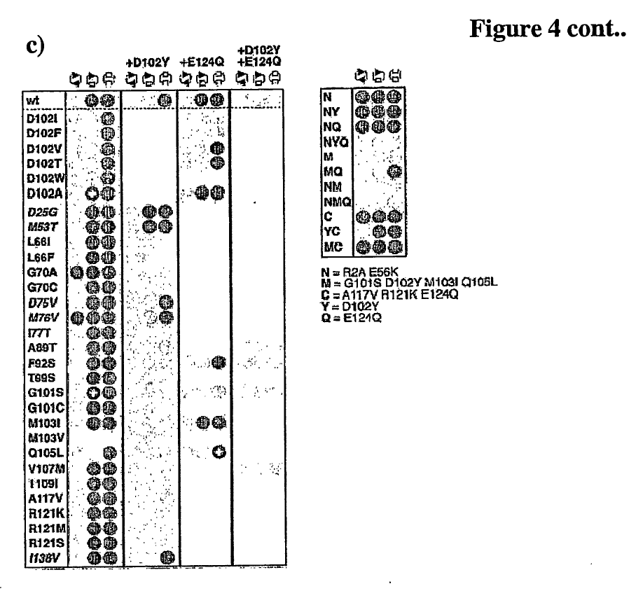

FIG. 4 shows

A) Plasmids used for in vivo screening for mutants. The repA gene product is required for initiation of replication at the pSC101 origin. See Arnold et al. (1999) for further details. Pgal(res×res) is shown. In pGal(res×I), res A has been replaced by a fragment containing site I, and in pGal(I×I), both res sites have been replaced by site I fragments. PStr(I×I) is similar to pGal(I×I), but the sequences containing the galK gene are replaced by sequences conferring resistance to tetracycline and sensitivity to streptomycin (see Materials and Methods section).

B In vivo properties of resolvase mutants. The colour of colonies on MacConkey agar plates is shown. Red (dark shaded circles) signifies no resolution activity, pale yellow (open circles) signifies full resolution, and pink (pale shaded circles) signifies slow resolution. Some mutants gave mixtures of colonies of different colours (shown as a sectored circle). Detection of weak activity of some mutants on certain substrates was variable, depending on factors such as colony density (dark shaded circles marked with a+sign).

C) Results of further experiments on the in vivo properties of resolvase mutants. Resolvases with single activating mutations, and their combinations with D102Y, E124Q, or both. The expression plasmids were selected as described in the text, or created by exchanging appropriate restriction fragments from different mutants. Some mutations (italicized) are not activating according to the results given in this Figure, but nevertheless contribute to hyperactivity of the originally isolated mutant. All D102 single mutants that are not shown here behave as wild-type resolvase.

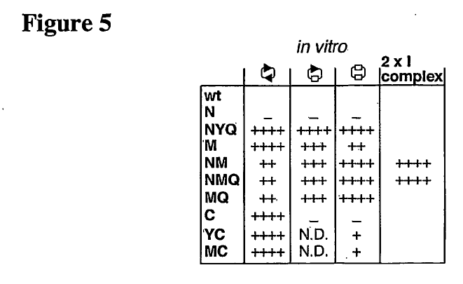

FIG. 5 shows

A) Summary of in vitro properties of some multiple mutants of resolvase. −=no activity. Higher activity is indicated by more+signs. The in vitro activities of D102Y, E124Q, and D102Y E124Q mutants are described in Arnold et al. (1999).

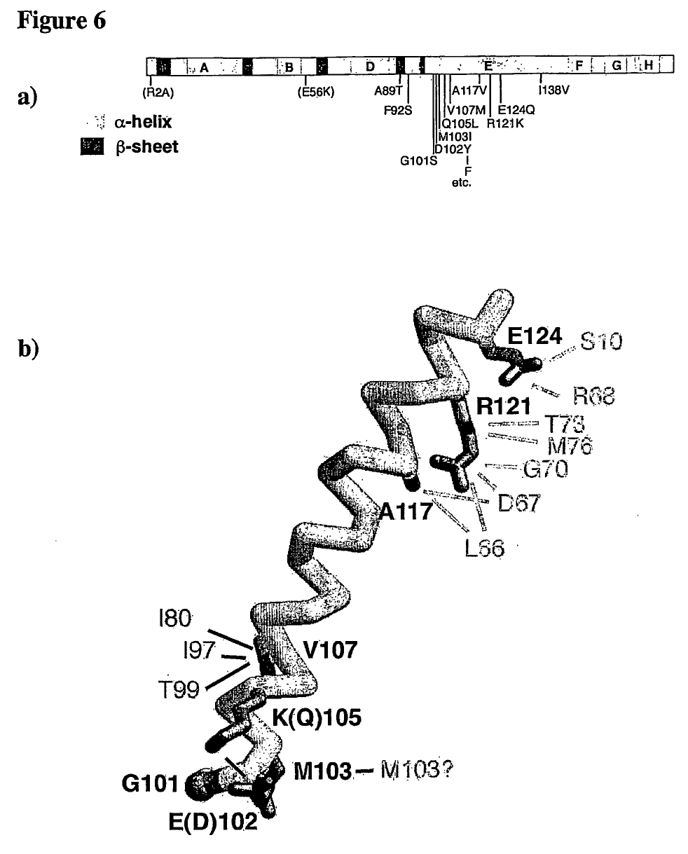

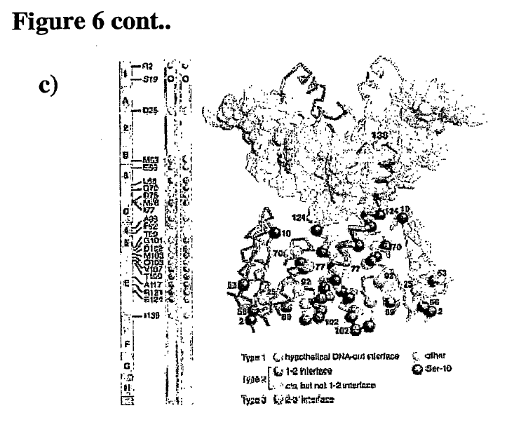

FIG. 6 shows

A) The location of the mutants which have been carried out by the present inventors.

B) Residues 100-125 of resolvase subunit A (Yang and Steitz, 1995), containing the N-terminal section of the E-helix and the immediately preceding residues, are shown in backbone representation. The view is from the same angle as in FIG. 2a. The sidechain of D102 is shown, and the sidechains of other residues mutated in the hyperactive proteins are also shown. Interactions of these residues are denoted by the thick lines.

C) Positions of the activating mutations (‘REG residues’). The γδ resolvase-DNA co-crystal structure (Yang and Steitz, 1995) is shown with the DNA spacefill representation, and the backbones of subunits A and B. The E-helix backbones are thicker. The α-carbons of REG residues are shown as spheres. Some residues are numbered. To the left, the Tn3 resolvase primary sequence is cartooned as a bar, with predicted α-helix unshaded, and β-sheet shaded. The secondary structure elements are designated as in Yang and Steitz (1995). The positions of REG residues are indicated. The residue type is indicated by spheres, as in the crystal structure image.

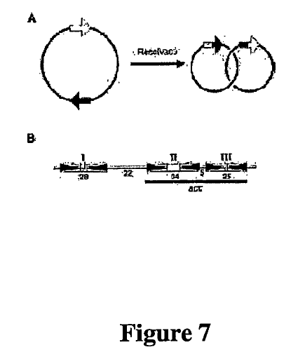

FIG. 7 shows in diagrammatic form resolvase-mediated site-specific recombination.

A) Resolvase catalyses recombination between two res sites (arrows) in head-to-tail orientation in a supercoiled plasmid. The product is a simple catenane, the two circles of which are unlinked in vivo by a Type II topoisomerase (not shown).

B) The recombination site res. The boxes represent binding sites for resolvase. Strand exchange takes place at the centre of binding site I, which is cleaved as indicated by the staggered line. The imperfectly repeated 12 bp sequences at each end of the three resolvase-binding sites are shown by arrowheads and shading. The sequence containing binding sites II and III is referred to in the text as acc. Lengths of DNA segments (bp) are indicated.

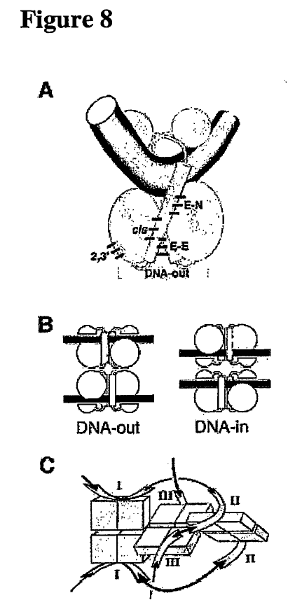

FIG. 8 shows resolvase-DNA complexes.

A) Cartoon of the γδ resolvase-site I co-crystal structure of Yang and Steitz (1995) (see also FIG. 6), indicating the positions of interfaces discussed in the text. The subunit-structure is shown as tripartite: the N-terminal subdomain (approximately residues 1-98; large oval), the E-helix (residues 103-136; cylinder), and the C-terminal domain (residues 148-183; small sphere). The 1-2 dimer interface is formed by contacts between residues of the two E-helices (labelled E-E), and contacts between residues of an E-helix and the N-terminal subdomain of the partner subunit (labelled E-N). The approximate position of the hypothetical ‘DNA-out’ dimer-dimer interface is shown as a bar.

B) Hypothetical interactions of two resolvase dimer-site I complexes, as may be required for synapsis and catalysis. The DNA is shown as a thick black line, and resolvase as a simplified version of the cartoon in A. C. A current model of the recombination synapse (Sarkis et al., 2001; Figure adapted from Rowland et al., 2001). The N-terminal domains of resolvase dimers are represented by ‘dominoes’ (C-terminal domains are not shown). The catalytic tetramer is bound at the paired site Is.

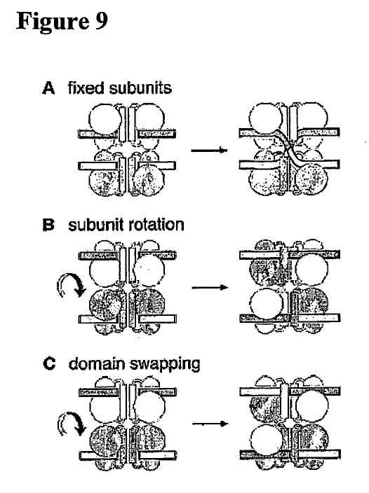

FIG. 9 shows current models for strand exchange by resolvase and related serine recombinases. The site I DNA is represented by grey bars, and the resolvase dimers are cartooned as in FIG. 2B. Each diagram on the left shows the hypothetical intermediate after cleavage of the four DNA strands; on the right, the DNA has been rearranged and ligated (i.e recombinant). For the fixed subunits model (A), a DNA-in tetramer is assumed, whereas for the subunit rotation (B) and domain swapping (C) models, a DNA-out tetramer is assumed.

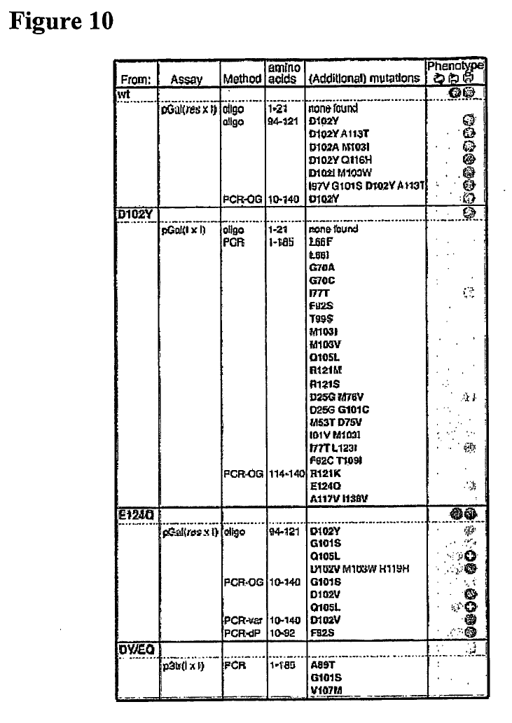

FIG. 10 shows hyperactive mutants of Tn3 resolvase. The ‘template’ for mutagenesis is given in the left-hand column (‘DY/EQ’ indicates the D102Y E124Q double mutant). The test plasmid used to assay resolution activity is given in the second column; the mutants were selected for their higher activity on that substrate than the template resolvase. The method used to create the mutant library is given in the third column; oligo, by cloning ‘spiked’ oligonucleotides; PCR, standard PCR amplification with Tag polymerase; PCR-OG, PCR with 8-oxo-dGTP in the reaction mixture; PCR-dP, PCR with dPTP in the reaction mixture; PCR-var, PCR with biased concentrations of the four standard dNTPs. Usually, only part of the resolvase ORF was subjected to mutagenesis (as stated in the ‘amino acids’ column). In all cases, the mutations shown are in addition to those of the template. Some mutants contained additional ‘silent’ DNA sequence changes (not shown). The mutations in bold face are sufficient to cause the observed phenotype. It may be that some of the ‘extra’ mutations (plain type) make an additional, minor contribution to hyperactivity; not all were fully tested separately (see FIG. 4). The colour of colonies on MacConkey agar plates containing galactose is shown in the ‘phenotype’ column. The substrate is indicated by the symbols at the top: from left to right, pGal(res·res), pGal (res·I), and pGal(I·I). Red (dark shaded circles) signifies no observable resolution activity, pale yellow (open circles) signifies full resolution, and pink (pale shaded circles) signifies partial resolution. Detection of weak activity with some mutant-substrate combinations was variable, as indicated by a dark shaded circle with a white+sign.

### MATERIALS AND METHODS

Mutagenesis

Designed mutations were introduced by cloning appropriate double-stranded synthetic oligonucleotides into pAT5 (Arnold et al., 1999), or pMA5811, which was derived from pAT5 by deletion of an EcoRV-NruI fragment. Random mutations were created using synthetic oligonucleotides as described in Arnold et al. (1999), or by the polymerase chain reaction. Primers flanking the complete resolvase ORF of pAT5 or pMA5811 were used to amplify the fragment, and mutagenesis was caused by biasing the proportions of the dNTPs (Fromant et al., 1995), or by introduction of 8-oxodGTP or dPTP nucleotides (Zaccolo et al., 1996). Appropriate restriction digest fragments from mutagenized DNA were cloned into pAT5 or pMA5811 to create libraries of mutants which were screened as described below.

Screening and Selection

Resolvase expression plasmids, in vivo expression, and the GalK-based screening method were as described in Arnold et al., (1999). pGal(res×res), pGal(res×I), and pGal(I×I) were described by Arnold et al. as pDB34, pDB37, and pDB35 respectively. Typically, between 1 000 and 10 000 candidate mutants were screened. The numbers were limited either by the diversity of the library, or by the screening procedure, in which ‘white’ colonies could not be picked reliably when there were more than ˜1 000 colonies on a single 8 cm diameter MacConkey agar plate. Some mutants were selected by a method in which resolution of a test plasmid causes loss of tetracycline resistance, but confers resistance to streptomycin. In the test plasmid pStr(I×I) (=pMA5531), the galK gene of pGal(I×I) was replaced by sequences containing a gene for tetracycline resistance, and the strA (rpsL) gene, encoding the wild-type ribosomal S12 protein from pABS12. When highly expressed, S12 causes streptomycin-sensitivity in strains of E. coli that are normally resistant due to a mutation in the chromosomal copy of this gene. Agar plates containing streptomycin and kanamycin therefore select for loss of the plasmid-encoded strA gene by recombination between the two site Is. Libraries of pAT5 containing mutant resolvase ORFs were used to transform E. coli strain DS941/pStr(I×I). Liquid cultures (LB medium) were grown without selection for variable time intervals before spreading aliquots on L-agar plates containing kanamycin and streptomycin (200 μg ml⁻¹). Mutant versions of pAT5 were isolated from colonies that appeared at early time points.

Results

Mutation Strategies

The point mutation D102Y allows Tn3 resolvase to recombine a res×site I substrate. The double mutant D102Y E124Q can slowly recombine a site I×site I substrate, although it is still greatly stimulated by the presence of acc (in a res×res or res×site I substrate) (Arnold et al., 1999). The present inventors therefore adopted three approaches to find other activating mutations of resolvase: (A) random mutagenesis of the catalytic domain of resolvase; (B) mutation of residue D102 to all other amino acids; (C) random mutagenesis of resolvases which already-contained D102Y, E124Q, or both mutations. The inventors then observed the effects of combining activating mutations with each other and/or with mutations at the 2,3′ interface.

Random Mutagenesis of the Resolvase Catalytic Domain

Libraries of resolvase expression plasmids mutagenized throughout the catalytic domain (residues 1-140) by PCR-based methods (see Materials and Methods), or between residues 94 and 121 with oligonucleotides (Arnold et al., 1999), were screened for resolution of pGal(res×I) in vivo, by an assay in which resolution of a test plasmid, with a gene for GalK flanked by two recombination sites, is detected by formation of white (gaIK⁻) rather than red (galK⁺) colonies on MacConkey indicator agar plates (Arnold et al., 1999; FIG. 4a). Sequencing of the resolvase expression plasmids from white colonies identified several hyperactive mutants, all of which were altered at residue 102 (Table 1). The present inventors noted that the single mutant M103I was erroneously stated to be hyperactive in Arnold et al. (1999); re-sequencing of the expression plasmid revealed an additional mutation D102A. M103I does not show detectable hyperactivity in the MacConkey assay, nor does it when combined with E124Q (see above; FIG. 4b). However, the D102A M103I and D102T M103T double mutants are more hyperactive than the corresponding D102 single mutants (see below). The G101S and Q105L single mutants were later found to be hyperactive (FIG. 4b; see below), though they were not recovered from screens of mutagenized wild-type resolvase, probably because their resolution of pGal(res×I) was insufficient to produce distinctly paler single colonies amidst many red colonies.

Saturation Mutation of D102

Residue D102 was mutated to all 19 other amino acid residues, by cloning synthetic oligonucleotides into the resolvase ORF of pAT5. The mutants were assayed as described above; the results are summarized in FIG. 4b. All 19 mutants resolved pGal(res×res) which has two full res sites. pGal(I×I), a plasmid with no acc, i.e. just two copies of site I, was not resolved detectably in this assay by any D102 mutant. pGal(res×I) was resolved efficiently by the mutants D102Y, D102F, and D102I. D102W, D102V, and D102T had lower activity on pGal(res×I), as indicated by a pinker colour of the colonies in the assay, D102A had barely detectable activity, and all other D102 mutants did not have detectable activity (i.e. red colonies). The mutant D100Y was also tested, but was not hyperactive (see Discussion).

To assess further the effects of the activating D102 substitutions, some of them were combined with E124Q, and the properties of the double mutants were compared (FIG. 4b). These results suggest that the most potent activating mutations of D102 are to F, Y, or I.

Random Mutation of D102Y, E124Q, and D102Y E124Q ORFs

The entire D102Y resolvase ORF was subjected to random mutagenesis by PCR-based methods. Mutants which resolved pGal(I×I) retained D102Y, and had the additional mutations A117V or R121K or E124Q (Table 1; FIG. 4b) or those shown as such in FIG. 4c. The original A117V isolates had a third mutation, I138V. The D102Y A117V double mutant was active on pGal(I×I), but less so than the original triple mutant. I138V was not hyperactive as a single mutant, and the D102Y I138V double mutant did not resolve pGal(I×I) (data not shown). The D102Y E124Q double mutant had been created previously by design (Arnold et al., 1999). Single mutants derived from these hyperactive multiple mutants were then assayed (FIGS. 4B and C). Of the single mutants isolated in this way, only Q105L was detectably hyperactive. The single mutants A117V, R121K, and E124Q resolved pGal(res×res), but resolution of pGal(res×I) or pGal(I×I) was undetectable.

E124Q resolvase was mutagenized by PCR, between residues 10 and 140. Libraries of mutants were screened for resolution of pGal (res×I), which is not resolved by E124Q itself. Second mutations which confer resolution activity were identified as F92S, G101S, D102V, D102Y, and Q105L (Table 1; FIGS. 4B and C). The G101S, D102Y, and Q105L (+E124Q) double mutants also resolved pGal(I×I). All of the derived single mutants except F92S had detectable activity on pGal(res×I) (FIG. 4b). Some other D102 mutants had increased hyperactivity when combined with E124Q (see above).

The entire resolvase ORF containing both D102Y and E124Q mutations was mutagenized by PCR. Because D102Y E124Q itself resolves pGal(I×I), an alternative method was used to select for E. coli containing resolvase mutants that were able to promote rapid resolution of a site I×site I plasmid pStr(I×I), thereby conferring streptomycin resistance (see Materials and Methods for details). Three mutants which rapidly resolved pStr(I×I) (and pGal(I×I)) had the additional mutations A89T, G101S, and V107M (Table 1 and FIG. 9). The derived double mutants A89T D102Y, G101S D102Y, and D102Y V107M all resolved pGal(I×I) (A89T D102Y less efficiently—pink colonies). A89T and V107M single mutants did not show detectable hyperactivity in the MacConkey plate assay (FIGS. 4B and C).

Combinations of Activating Mutations

The results described above indicated that some combinations of activating mutations were more effective than single mutations. The present inventors therefore tested resolvases with several designed combinations of mutations, by making ‘cassettes’ containing either four mutations of residues at or near D102 (G101S D102Y M103I Q105L; M-cassette), or three mutations nearer the C-terminus (A117V R121K E124Q; C-cassette). Further mutants were then created by combining the cassettes with D102Y, E124Q, or each other (FIG. 4b). The M-cassette mutant promoted efficient resolution of of all three pGal test plasmids. Combination of the M-cassette with E124Q (MQ) decreased activity on pGal(res×I) and pGal(I×I). The C-cassette mutant did not promote detectable resolution of any of the pGal plasmids, nor did the mutant containing both M- and C-cassettes (MC). Combination of the C-cassette with D102Y (YC) restored resolution of pGal(res×res), but the other pGal plasmids were not resolved.

Effect of Mutations at the 2,3′ Interface

Mutations of residues at the 2,3′ interface abolish recombination activity in γδ resolvase. Tn3 resolvase mutant in two 2,3′ interface residues (R2A and E56K; N-cassette; see Hughes et al., 1990) was likewise completely inactive, in vivo (FIG. 4b) and in vitro (unpublished results). The triple mutants R2A E56K D102Y (NY) and R2A E56K E124Q (NQ) were also inactive, but in striking contrast, the quadruple mutant R2A E56K D102Y E124Q (NYQ) was hyperactive; more so than the double mutant D102Y E124Q. The M and MQ multiple mutants (see above) were also combined with R2A E56K (N-cassette), creating NM and NMQ multiple mutants. These proteins efficiently resolved all three pGal test plasmids. Our random mutagenesis experiments identified one activating mutation of a residue close to the 2-3′ interface, M53T.

In Vitro Properties of Multiple Hyperactive Mutants

Several of the hyperactive resolvases were over-expressed and purified. Their in vitro activities are summarized in FIG. 5, and broadly agree with the phenotypes observed in vivo (FIG. 4b). The present inventors analysed the multiple mutant M resolvase and its 2,3′-defective derivative NM in detail. Both resolvases were active on a site I×site I supercoiled plasmid pTet(I×I) in vitro, but NM resolvase was significantly more active. About half of the substrate was recombined in 4 minutes, a rate similar to that of wild-type resolvase on the standard resolution substrate pTet(res×res) under similar conditions. Site I of res is functionally symmetric (Bednarz et al., 1990), so as expected NM resolvase gave about equal amounts of resolution and inversion products from pTet(I×I). There was no evidence of topological selectivity; a series of knots and catenanes, consistent with random collisions of sites, was formed from single pTet(I×I) molecules, as well as products of recombination between sites on separate molecules. Unexpectedly, acc sequences inhibited recombination by N and NM resolvases; recombination of pTet(res×I) was slow, and recombination of pTet(res×res) was even slower. These mutants do not use acc to impose selectivity. Resolution was not preferred over inversion, and the distributions of product topologies from all three pTet plasmids were similar; there was no evidence of a preferred 2-noded catenane product from pTet(res×I) or pTet (res×res). M and NM resolvases also promoted rapid intra- and intermolecular recombination between site Is on linear DNA molecules (unpublished results).

Discussion

The role of acc

Wild-type resolvase binds to a substrate containing two copies of site I, but does not catalyse any recombination. Acc sequences correctly positioned adjacent to both site Is (that is, a res×res substrate) are essential for efficient catalytic activity (Bednarz et al., 1990). Hyperactive mutants promote recombination between two site Is in the absence of one or both of the acc sequences. The ‘hyperactive’ resolvase mutants characterized in this study are gain-of-function mutants that can catalyse reactions not observed with the wild-type enzyme: res·site I or site I·site I recombination. Our observation that mutations at many residues can contribute to hyperactivity suggests that they act by disrupting or circumventing a natural regulatory mechanism which enforces acc-dependence, rather than by conferring an intrinsically new functionality. Possible ways in which they might do this are considered below. It is worth noting that ‘hyperactive’ mutants do not necessarily resolve a res·res substrate faster than wild-type resolvase; in fact, in vitro, the opposite is observed in some cases (J. H. et al., manuscript in preparation).

It was expected that hyperactive resolvase-mediated site I×site I recombination in plasmids would be topologically non-selective (see Arnold et al., 1999), because topological selectivity of wild-type resolvase involves its interactions with acc (see Introduction). More surprisingly, in vitro recombination by M, NM, and some other hyperactive mutant resolvases was inhibited by the presence of acc sequences in res×res or res×site I substrates, and the mutants do not use acc to specify a single product topology. Without wishing to be bound by theory, the present inventors speculate that subunits of these mutants bound at res tend to make inappropriate interactions with each other or with subunits bound at a partner recombination site, which inhibit recombination and disable the proper function of acc.

Since two acc resolvase complexes can make a stable synapse (Watson et al., 1996; Kilbride et al., 1999), our current hypothesis is that, in wild-type resolution, this two-acc synapse forms first, then makes contacts with resolvase bound at site I (probably using the 2-3′ interface, as in FIG. 8C). This process might simply bring the two resolvase-site I complexes together in an appropriate geometry for catalysis (‘recruitment’), or it might cause a conformational change in the catalytic resolvase subunits that is required for activity (‘stimulation’).

Activating Mutations

The present screens have been sufficiently thorough that the inventors are confident that all or nearly all the residues that can be mutated to give hyperactivity have been identified. They are all in the catalytic domain of resolvase, between amino acid residues 89 and 124 (except for the weakly enhancing mutation I138V) (see FIG. 6). This region comprises the last two strands of the β-sheet that forms the core of the catalytic domain, the N-terminal part of the E-helix, and short connecting loops. Many of the residues in this segment of the polypeptide sequence are involved in the interface between the two subunits of the resolvase 1,2-dimer.

Only three residues yielded hyperactive single mutants; G101, D102, and Q105. Of these, only the D102 mutants could promote complete resolution of pGal (res·I) in our in vivo assay, and all the hyperactive resolvases identified by random mutagenesis of the wild-type protein were altered at D102, G101, D102, and Q105 are all solvent-exposed residues, far from the active site of resolvase, and do not contribute to subunit interactions in any of the current γδ resolvase crystal structures (see Introduction). In γδ resolvase, residue 102 is a glutamate and residue 105 is a lysine. Residues 99-102 make a loop connecting the C-terminal strand of the β-sheet at the core of the catalytic domain to the long E-helix, which begins at residue 103 and makes a major contribution to the dimer interface. Multiple mutations confined to this part of the primary sequence (e.g. G101S D102Y) are sufficient to confer full independence from acc (that is, complete resolution of pGal(I·I)).

D102 is the only single residue mutant which can promote complete resolution of pGal(res×I). The long E-helix which is a major component of the dimer interface begins at residue 103, and residues 99-102 make a loop connecting the E-helix to the C-terminal strand of the catalytic domain core β-sheet (Yang and Steitz, 1995). D102 (E102 in γδ resolvase) does not contribute to any of the known resolvase interfaces (see Arnold et al., 1999, for more details). Mutating D102 to all other amino acids, remarkably, all 19 mutants resolved pGal(res×res). Seven mutants were observed to be hyperactive. The sidechains of the activating substitutions (Y, I, F, V, T, W, and A, in approximate order of decreasing effect) are all uncharged and hydrophobic, but other hydrophobic residues (M, L, and P) do not activate detectably. The mutation G101S, of the residue preceding D102, is also activating, and the double mutant G101S D102Y promotes complete resolution of pGal(I×I), with no acc.

Only one other single mutant, Q105L, was detectably hyperactive in the MacConkey assay. The equivalent γδ resolvase residue, K105, is within the E-helix, but apparently does not participate in the dimer interface. The nearby activating mutation V107M maps to γδ resolvase residue V107, whose sidechain is in a very different environment—deeply buried in the hydrophobic centre of the dimer. It interacts with residues in the N-terminal ‘subdomain’ (residues 1-100; see below) of its own subunit, and with the E-helix of the other subunit of the dimer.

The activating effects of all other mutations are only evident in combination with at least one other REG residue mutation. These combinations nearly always include a G101, D102, or Q105 mutation, and activation consists of enhancement of their hyperactive phenotype (the sole exception that we found is F92S E124Q). In two cases, two mutations in addition to D102Y were necessary for further activation. The complete set of mutations that we have shown to contribute to hyperactivity is listed in FIG. 4. In addition, we showed that R2A and E56K, mutations of residues at the 2-3′ interface, can together enhance hyperactivity of mutants that already have some site I·site I activity (FIG. 4B).

The sidechains of the three residues A117, R121, and E124 are on the same face of the E-helix and contact the partner subunit of the dimer, at or near its presumptive catalytic site (Yang and Steitz, 1995;). This group of interactions is present only once in the crystal structure of the DNA-bound 1,2 dimer, being one of its most obvious asymmetric features. The mutations A117V, R121K, and E124Q are all conservative changes, whose activating effect (in Tn3 resolvase) is only manifested in the presence of at least one other activating mutation (D102Y in all cases tested).

Three other mutations, A89T, F92S, and I138V, had an enhancing effect when combined with other activating mutations (FIG. 4b). A89 (S89 in γδ resolvase) is on the surface of the resolvase dimer. It has been noted to be on the putative interface of dimers in the ‘DNA-out’ tetramer (Sarkis et al., 2001). The F92 sidechain makes various hydrophobic interactions, including one with the E-helix (L111) of its own subunit. Residue I138 (V138 in γδ resolvase) is the second residue beyond the C-terminal end of the E-helix; its sidechain contacts a deoxyribose of the DNA backbone in the minor groove. Possibly the enhancing effect of the I138V mutation is due to suppression of an undesirable interaction, as suggested for mutations at the 2,3′ interface (see below).

The identification of multiple mutants with stronger hyperactivity led the present inventors to construct two ‘cassettes’ with groups of mutations which were close to each other in the primary sequence. Resolvases with all four mutations G101S, D102Y, M103I, and Q105L promoted rapid recombination of site I×site I plasmids, in vivo and in vitro. In contrast, catalytic activity of resolvases with the three mutations A117V, R121K, and E124Q was severely reduced.

The acc-independent activity of hyperactive mutants is stimulated by additional mutations at the 2,3′ interface. The 2,3’ interface is clearly therefore not required for the catalytic steps of recombination (see also Grindley, 1993; Murley and Grindley, 1998; Sarkis et al., 2001). A recent model for the synaptic complex (Sarkis et al., 2001) proposes that resolvase dimers bound at site I make 2,3′ interactions with subunits in the rest of the synapse. Again without wishing to be bound by theory, the present inventors speculate that this interaction is pivotal to the mechanism of activation of catalysis in the natural system, but it may be superfluous and mildly inhibitory for mutants that do not require acc.

The DNA invertases Gin, Cin, and Hin are quite closely related to Tn3 resolvase; the amino acid sequences can be aligned along their entire lengths. These recombinases have been screened for activating mutations which abolish requirement for an accessory DNA ‘enhancer’ segment, or the protein FIS that binds to it (Haffter and Bickle, 1988; Klippel et al., 1988; Johnson, 2002). The idea that the effects of the invertase and resolvase activating mutations are analogous has been noted previously (Arnold et al., 1999). The invertase mutations map to the following residues of Tn3/γδ resolvase: A74, 177, Q78, V90, I97, V107, T109, A115, A117, E124. We identified resolvase activating mutations at five of these ten residues (underlined).

Mutations at a surprisingly large number of resolvase residues (20 out of 185; FIG. 4C) were found to cause or enhance hyperactivity. We will refer to these as REG residues (involved in regulation). We have probably identified most of the REG residues, but only a fraction of all potential activating mutations, because many codon changes are inaccessible by any practicable method of random mutagenesis. The REG residues are all in the catalytic domain of resolvase, but otherwise do not fall neatly into one obvious structurally distinct category.

The resolvase REG residues can be grouped into four types (FIG. 6): (1) residues at the hypothetical DNA-out interface; (2) residues involved in interfaces between subunits or structural domains of the 1-2 dimer; (3) residues at the 2-3′ interface; (4) others.

Type 1: Residues at the Hypothetical DNA-Out Dimer-Dimer Interface

Our results strongly support the idea that the catalytic unit is a DNA-out synaptic tetramer (FIG. 8B). A number of REG residues are on or close to the hypothetical interface required for the formation of this tetramer. These ‘Type 1’ residues include A89, G101, D102, M103, and Q105 (the interface is described in more detail by Sarkis et al., 2001). Of these residues, only M103 makes inter-subunit interactions in existing crystal structures of resolvase (Table 1).

Remarkably, although D102 seems to have a crucial role in regulation, all nineteen D102 mutants fully resolve pGal(res·res). The D102Y single mutation is sufficient to permit a low level of site I·site I recombination in vivo. The sidechains of the seven activating substitutions of D102 (Y, I, F, V, T, W, and A, in approximate order of decreasing effect) are all uncharged and rather hydrophobic, but other bulky hydrophobic sidechains (M, L, and P) do not activate detectably. The tendency towards uncharged, more hydrophobic sidechains in the activating mutations of D102 and the other Type 1 REG residues is consistent with the idea that they stabilize the DNA-out tetramer. In vitro, γ resolvase with several mutations including G101S, E102Y, and M103I made a stable synapse, comprising two site Is and four resolvase subunits (Sarkis et al., 2001). Topological experiments with hyperactive resolvase mutants (Leschziner and Grindley, 2003) also support a DNA-out tetramer. Tn3 resolvase G101S D102Y and other similar multiple mutants (e.g., those with the M-cassette group of mutations) are fully hyperactive in vivo (FIG. 4), and rapidly recombine a site I·site I substrate in vitro (J. H. et al., manuscript in preparation), implying that the sidechains of the residues around D102 are not critical to any folded structures required for catalysis.

Interestingly, three of the Type 1 REG residues differ between Tn3 and γ≢7 resolvase (A/S89, D/E102, Q/K105). A simple hypothesis is therefore that stable synapsis of two resolvase-bound site Is normally depends on interactions of subunits bound at site I with subunits bound at acc, and the Type 1 mutations remove this dependence by stabilizing the DNA-out dimer-dimer interface. It is intriguing that none of the reported activating mutations of the DNA invertases are at residues likely to contribute to this interface (see above). Perhaps this is because the wild-type invertase tetramer is constitutively more stable, as is suggested by direct observation of synapses between Hin dimer-DNA complexes (Heichman and Johnson, 1990). In contrast, no synapses of site I mediated by wild-type resolvase were observed in similar experiments (Watson et al., 1996)

An alternative proposition, that some of the Type 1 mutations cause hyperactivity by altering the properties of a ‘hinge’, is discussed below under ‘The mechanism of strand exchange’.

Type 2: residues involved in subunit/domain interfaces The 1-2 dimer interface is intricate and strikingly hydrophobic. It involves interactions of more than 20 residues, as summarized in Table 1 (data based on the co-crystal structure of Yang and Steitz (1995); the other crystal structures show some subtle differences in the structure of the interface). There are two kinds of contacts between the subunits: (1) between the two E helices, and (2) between an E-helix and the N-terminal ‘subdomain’ of the partner (residues 1-98) (Table 1; see also FIG. 8A). Most of these contacts involve a known REG residue, and in some cases both contacting sidechains are of REG residues (Table 1). The REG residues at the dimer interface are L66, G70, M76, M103, V107, T109, A117, R121, and E124. Some residues at the active site also contribute to the dimer interface, including S10, the catalytic nucleophile.

The N-terminal subdomain appears to have some structural autonomy, and a fragment comprising residues 1-105 of γδ resolvase is properly folded in solution (Pan et al., 2001). The subdomain is fixed into the crystallographic 1-2 dimer by its participation in the dimmer interface, and additionally by a ‘cis’ interface involving contacts with the E-helix of its own subunit (FIG. 8A). The role of the N-terminal subdomains in strand exchange is discussed in the next section. Six REG residues lie on the cis interface: L66, M76, I77, F92, T99, and V107. The underlined residues also contribute to the 1-2 dimer interface. Five of the ten reported positions of invertase activating mutations (see above) map to resolvase residues on the cis interface, including the REG residues I77 and V107.

All but one of the hyperactive resolvases with Type 2 mutations also contain a Type 1 mutation; the exception is the feebly hyperactive F92S E124Q. F92 is on the same-strand as the Type 1 residue A89, and mutations of F92 might therefore have some Type 1 character. We speculate that, in general, Type 2 activating mutations are effective only in the presence of a Type 1 mutation stabilizing the DNA-out interface (see ‘The mechanism of strand exchange’, below). The γδ resolvase single (Type 2) mutant E124Q is hyperactive in our assay (Arnold et al., 1999), suggesting that the DNA-out interface of γδ resolvase may be more stable than that of Tn3 resolvase.

The Type 2 activating mutations are generally quite conservative. Even so, some of them have large effects on recombination activity. The point mutations G70A and M76V abolish activity of resolvase even on pGal(res·res) (FIG. 4C), despite their activating properties in the presence of additional mutations. Similarly, the C-cassette mutant, with three Type 2 mutations, is inactive, but activity on pGalx(res·res) can be restored by the additional mutation D102Y (FIG. 4B).

We suggest two mechanisms by which Type 2 mutations might enhance acc-independent catalysis of strand exchange.

First, they might alter the configuration or accessibility of the active site residues. Second, they might facilitate rearrangement of the protein by destabilizing interfaces.

The sidechains of the three Type 2 residues A117, R121, and E124 (mutations of which together constitute the ‘C-cassette’) are on the same face of the E-helix, at consecutive turns. Their contacts, which might be disrupted by the mutations, include the presumptive active site residues S10, D67, R68, and R71 of the partner subunit (FIG. 6, Table 1). The sidechain configurations of D67, R68, and R71 might also be perturbed by mutations of neighbouring residues, e.g. L66 and G70. Similarly, the sidechain of the Type 2 residue D75 interacts with putative active site residues of its own subunit, including R45 and R71. The configuration of active site residues varies among the crystallographic structures of γδ resolvase, and implications of these variations for catalysis have been discussed (Rice and Steitz, 1994b).

Perturbation of the active site sidechains might somehow cause activation, but we do not put forward any more detailed speculation because very little is known about the interactions of the active site with the DNA substrate. Many of the Type 2 residues contribute to the 1-2 dimer interaction, so mutations of these residues might destabilize the dimer interface. Some hyperactive mutants have increased monomeric character in vitro (Arnold et al., 1999; J. H. et al., manuscript in preparation), and ‘hyperactive’ behaviour in Hin invertase and γδ resolvase has been elicited in vitro by the addition of detergents to reaction mixtures (Haykinson et al., 1996; M. R. B. and N. D. F. Grindley, unpublished results), supporting the idea that weakening of the dimer interface may be significant. It is striking that all but one of the Type 2 REG residues (the exception being D75) are involved in contacts between the N-terminal subdomain and the rest of the dimer.

Three of these residues (I77, F92, and T99) are on the cis interface, but do not make any contacts across the dimer interface. Perhaps the most inclusive interpretation of activation by the Type 2 mutations is therefore that they destabilize the docking of the N-terminal subdomains with the remainder of the catalytic tetramer, a scenario consistent with a ‘domain swapping’ mechanism for strand exchange (see below).

Type 3: Residues at the 2-3′ Interface

Mutants with some site I·site I activity are activated further by mutations that are known to destabilize the 2-3′ interface (R2A and E56K; FIG. 4B). The 2-3′ interface is therefore not required for the catalytic steps of recombination (see also Grindley, 1993; Murley and Grindley, 1998; Sarkis et al., 2001). However, it is necessary for catalysis by wild-type resolvase, and hyperactive mutants that still require one full res site (e.g. D102Y resolvase; FIG. 4B). Our screens identified one activating mutation of a residue close to the 2-3′ interface (M53T). This mutation apparently does not abolish the interface, because the single mutant resolves pGal (res·resi (FIG. 4C); in contrast, the single mutants R2A and E56K are inactive (Wenwieser, 2001; S-J.R., unpublished results). Hypothetically, mutations of residues at the 2-3′ interface could stimulate activity by mimicking effects of the acc-resolvase moiety of the synapse. However, a simpler explanation for the observed enhancement of site I·site I activity by the ‘interface-knockout’ mutations R2A and E56K (FIG. 4B) would be that they prevent dimer-dimer interactions which have become unnecessary and counter-productive.

Type 4: Other Residues

Only two REG residues, D25 and I138, do not fall into the above three groups. We found the mutation D25G in two separate contexts (FIG. 10), with D102Y M76V or D102Y G101C. All three substitutions in the D25G M76V D102Y triple mutant are required for activity on pGal(I×I) (FIG. 4C, and data not shown). D25 is a surface residue that does not participate in any known subunit interactions, but a current synapse model implies that it might be involved in a further dimer-dimer interface (Sarkis et al., 2001). The mutation I138V was found in combination with D102Y A117V; the derived double mutant D102Y A117V was less active than the triple mutant on pGal(I·I). Residue I138 (V138 in γδ resolvase) is the second residue beyond the C-terminal end of the E-helix; its sidechain contacts a deoxyribose of the DNA backbone in the minor groove. It is the only residue close to the hypothetical ‘DNA-in’ interface (FIG. 2B) to have emerged from our screens. However, many recent experiments argue against a catalytic DNA-in tetramer (see above, and Akopian et al., 2003). Possibly, the enhancing effects of both D25G and I138V are due to suppression of unfavourable interactions, as is suggested above for Type 3 mutations (at the 2-3′ interface).

The Mechanism of Strand Exchange

Three models for the mechanism of strand exchange by serine recombinases are currently in vogue.

Rice and Steitz (1994a) proposed that strand exchange would be accomplished without large scale movements or conformational changes of the resolvase subunits. This type of mechanism has been established for the tyrosine recombinases Cre and FLP (reviewed by Chen and Rice, 2003). The two site Is bound by resolvase would make a ‘DNA-in’ synapse, with the resolvase catalytic domains on the outside (FIG. 9A) (see also Merickel et al., 1998). This model is not supported by the results presented here, which favour a DNA-out synaptic tetramer. The model is also difficult to reconcile with DNA topological studies on strand exchange (Stark and Boocock, 1994; Mcllwraith et al., 1997).

Stark et al. (1989) proposed that, following cleavage of the four DNA strands, the ends are exchanged by a 180° rotation of a pair of resolvase subunits in a ‘DNA-out’ tetramer (although a DNA-in tetramer could also be compatible with the model) (FIG. 9B). Subunit rotation is a simple model that is consistent with all available topological and biochemical data, but there is still no solution to the problem of how rotation of the subunits could be accomplished without catastrophic dissociation of the four half-sites comprising the resolvase-cleaved DNA complex (Grindley, 2002). Mutations of residues at the dimer interface (Type 2) might activate by facilitating the dissociation of this interface, as would be required for rotation.

In the ‘domain swapping’ model (Grindley, 2002; FIG. 9C), the DNA moves as in subunit rotation, but only the N-terminal parts of the resolvase subunits rotate through 180°. The subunits that swap domains maintain their association with the remainder of the complex by static interactions of their E-helices. A DNA-out catalytic tetramer (FIG. 8B) is integral to this model. The ‘hinge’ that would be required in two subunits of the tetramer (FIG. 9C) is proposed to be in the loop at the N-terminus of the E-helix, residues 99-102. Intriguingly, the most effective Type 1 activating mutations (of residues D102 and G101) are in this loop. These mutations might have a dual effect, stabilizing the dimer-dimer interface and facilitating the operation of the hinge. Type 2 mutations might enhance hyperactivity by weakening the interactions of the N-terminal subdomain sufficiently to allow rotation in the absence of the normal activating stimulus from acc.

In summary, a scenario that explains the properties of the activating mutations is as follows.

For wild-type resolvase, the catalytic DNA-out tetramer must be stabilized by interactions with subunits bound at acc, in a synapse as in FIG. 8C. The primary obstacle to acc independence, instability of the tetramer, is overcome by Type 1 mutations. Two or more Type 1 mutations are sufficient to confer full activation in our assay, by increasing the stability of the tetramer and/or by facilitating the operation of the hypothetical 99-102 hinge.

Alternatively, a Type 1 mutation conferring site I·res activity can be complemented by Type 2 mutations to generate site I·site I activity, by destabilizing tetramer-internal interfaces (facilitating subunit or domain rotation), or by altering the configuration of the catalytic site.

Finally, Type 3 or Type 4 mutations can enhance site I·site I activity by inhibiting unfavourable interactions at the surface of the catalytic tetramer. This interpretation implies a ‘recruitment’ role for the acc-resolvase complex (see above) which can be bypassed by the tetramer-stabilizing Type 1 mutations. The effects of the Type 2 mutations, many of which are buried within the resolvase dimer, cannot be explained easily by a pure recruitment model, and suggest that acc may also be required to induce conformational changes associated with catalysis by wild-type resolvase. However, conformational changes facilitated by Type 2 mutations might also stabilize the catalytic tetramer; whether or not this is so remains to be established.

Our results are consistent with the domain swapping model for strand exchange; this model, unlike subunit rotation, readily explains the activating mutations of Type 2 REG residues that contribute exclusively to the cis interface (I77, F92, and T99).

Development of Hybrid Recombinases

Hybrid recombinases have been developed which comprise a Tn3 resolvase catalytic domain linked to a zinc-binding domain, Zif268. All the recombinases tested comprise residues 1-144 of the resolvase mutant “RMMD+”, which has the following changes from wild-type; R2A, E56K, G101S, D102Y, M103I, Q105L. The first two mutations are to the “2,3′” interface, and the other 4 are “activating” mutations. In all cases the Zif268 domain has the wild-type sequence starting from residue 2 as given in the crystal structure paper (N. P. Paveletich and C. O. Pabo, Science 252, 809-817 (1991). Between residue 144 of resolvase and residue 2 of Zif268 there is a “linker” sequence. The sequences tested are shown in FIG. 2b. All proteins show activity; the most active in E. coli was the one with the linker marked with a big asterisk.

The sites used are also shown in FIG. 2a. The relevant ones are those marked Z0, Z+2, etc. They comprise two invariant 9 bp motifs recognized by Zif268 (pale blue boxes with three little arrows inside), flanking a central invariant sequences made up of at least 13 bp of sequence from the centre of res site I (darker pink shading), and some varied “spacer” basepairs which change the distance between the two Zif268-bining motifs. The site marked with the big asterisk (Z+6) gave the highest recombination in E. coli (about 75% of substrate recombined after about 20 generations of growth). Z+4, and Z+8, 10, 12 also showed activity. The Z0 and Z+2 sites were inactive.

All combinations of hybrid proteins and sites shown in FIGS. 2a and b have been tested in E. coli. The Z0 and Z+6 sites, and two hybrid proteins corresponding to the linker marked with the asterisk and the one at the top of the list, have been tested in vitro. In vitro, the Z+6 site recombines much better than the Z0 site, which is almost inactive.

Based on the detailed knowledge of Tn3 site-specific recombination and mutants described above, the intention is to design novel systems for promotion of DNA rearrangements in for example higher eukaryote cells. It is first necessary to test and show that resolvase mutants can recombine substrates containing two copies of a minimal 28 bp recombination site (‘site I’;), in mammalian cell lines. The methods to be used are quite well established, Groth, et al. 2000 and Schwikardi and Dröge, 2000. Experiments may initially be in two or three standard cell lines, for example COS-1, 3T3, or 293 cells. A mutant resolvase will be expressed in the mammalian cells from a suitable plasmid derived from available vectors, with a standard promoter such as the SV40 early or CMV immediate early viral promoters, and a transcription terminator/polyadenylation signal. Further experiments may involve quantitative estimation of the extent of recombination, using constructs in which recombination changes expression of a reporter gene (eg. luciferase and/or GFP). To determine the cellular localization of the mutant resolvases, it is possible to create fusions with green fluorescent protein (GFP) by established methods, and analyse cells expressing the fusion proteins by microscopy. These fusion proteins will also allow easy determination of transfection efficiency. The present inventors have already demonstrated full recombination activity by resolvase-GFP fusion proteins in vitro and in E. coli. If it proves to be desirable, it is possible to attach a nuclear localization signal to the resolvase coding sequence.

It is then possible to compare the efficiencies of existing hyperactive resolvase mutants, to identify the features of the recombinase that are most important for efficient recombination at minimal sites in the cell lines, and to create potentially improved versions of the system. Further optimization of recombination activity may be achieved by selection strategies, which will be easily adapted from established methods for selection of resolvase mutants in E. coli see above. For example, it is possible to make a construct that contains a gene for a hyperactive resolvase, adjacent to a pair of recombination sites flanking a marker gene. Libraries of mutants in the resolvase ORF may be created, e.g. by PCR mutagenesis and in vitro ‘shuffling’. Cassettes which recombine upon transfection into mammalian cells can be recovered by PCR amplification, and are likely to encode an active resolvase. The sequences encoding the active resolvases can be subjected to further mutagenesis if required. The same plasmid constructs can be used for selection experiments in E. coli.

The natural res site I is functionally symmetrical, so either excision or inversion can occur in a substrate with two sites. Alteration of the 2 bp sequence at the centre of site I can break this symmetry, so that only one type of event (resolution or inversion) is allowed; this restriction might be desirable for most biotechnology applications, and it is straight-forward to test the properties of such sites in cell lines. It is predicted that other simple mutations in the sequence of site I might increase efficiency of recombination in the cell lines, and reduce the likelihood of reversal of the rearrangement by a second round of recombination.

Successful demonstration of efficient recombination following co-transfection may be followed by more stringent tests requiring excision of a marker gene which is inserted into the chromosomal DNA at random sites. This will more accurately reflect the situation in applications of the system, when the sequences of interest are typically associated with nuclear chromatin. Random integrants could be created by transfection of a suitable plasmid substrate followed by selection for a gene encoded by the plasmid, for example neomycin resistance. An intermediate approach which might prove to be very useful is to construct substrate plasmids containing sequences from Epstein-Barr virus, which have been shown to be located in the cell nucleus, chromatin-associated, and replicated with the chromosomes, thus being very good models for chromosomal DNA. These plasmids can be scored for recombination by isolation of cell DNA and transformation of E. coli.

It is also possible to optimize resolvase-Zif268 hybrid recombinases as exemplified for activity in mammalian cells, using methods analogous to those described above for the intact resolvase protein. The structure of Zif268 bound to DNA has been solved, (Elrod-Erickson, et al., 1996) and it is the focus of studies aiming to create engineered zinc finger proteins that can recognize any defined short DNA sequence. The properties of new variants of the hybrid recombinase will be studied first in E. coli, and in vitro, to allow for more detailed analysis and troubleshooting; suitable candidates will then be tested in mammalian cells. Further improvements in activity should be achievable by the application of mutagenesis followed by selection methods as described above. If these prototype recombinases are functional, it should be feasible to create analogous systems which recombine at a wide variety of synthetic sites, by replacing the natural Zif268 domain with known mutant versions that recognize different sequences (Chou & Isalan, et al., 2000 and Wolfe et al., 1999). This would create the potential for applications of site-specific recombination technology where two or more recombination events can be promoted independently in the same cell.

A straightforward extension of this approach is to attempt to target site-specific recombination to natural sequences in genomic DNA. This would be achieved by replacing the Zif268 domain of the hybrid recombinase with an altered version engineered to recognize part of the target sequence. Serine recombinases are much more promising for this type of application than the tyrosine recombinases such as Cre; tyrosine recombinases are not obviously divisible into ‘catalytic’ and ‘DNA recognition’ domains, and require more homology between the recombining sites (6-8 bp, versus only 2 bp for serine recombinases). One application would be the targeting of a recombinase to a sequence in the human immunodeficiency virus (HIV) provirus. Excisive recombination between the two LTRs of the provirus (roughly 9 kbp apart) would eliminate it from the genome, thereby providing a potential basis for therapy. Additionally it may be possible to target other genomic sequences. One such application would be targeted integration of gene cassettes at bovine casein gene loci, with the aim of creating transgenic animals which can produce large quantities of pharmaceutically useful proteins (Wilmut et al., 1991).

Suitable sites for targeting will have a sequence resembling as far as possible the central basepairs of res site I, which are contacted by the N-terminal domain of resolvase and thus affect the efficiency of catalysis of strand exchange, flanked by sequences that can be recognized by one or two engineered versions of the Zif268 DNA-binding domain. Most potential target sequences will have insufficient dyad symmetry for strong binding by both subunits of a diner of a single hybrid recombinase. However, evidence from current studies indicates that strong binding by only one subunit of the resolvase dimer can lead to efficient recombination at a minimal site. A more sophisticated solution of this problem, if it turns out to be necessary, would be to express two versions of a hybrid recombinase, which could form heterodimers with appropriate sequence recognition properties. The Zif268 domain(s) of the hybrid recombinase may therefore be modified for optimal binding at one or both of these sequences, based on the latest published information (see Choo & Isalan, 2000). Sequence recognition could be improved if required, by established selection methods for zinc finger proteins (Isalan et al., 2001). Substrates containing two copies of the potential recombination site may be constructed and analysed as described above. In the case of targeted integration, efficiency will be improved by optimization of the sequence of the recombination site associated with the gene cassette to be integrated. It may also be possible to reduce or eliminate the possibility of reversal of the integration reaction, by design of the cassette-associated site, or by incorporating features from the ΦC31 integration-specific serine recombinase system, which is also being actively studied for potential uses in mammalian cells (Groth et al, 2000).

TABLE 1

| | E-helix Residue | | | |
|---|---|---|---|---|
| | E-helix A → | | E-helix B → | |
| | → cis | → trans | → cis | → trans |
| | (to A) | (to B) | (to B) | (to A) |
| M103 | — | — | S98 | M106 |
| G104* | D84 T99 | — | T99 | — |
| Q105 | — | — | — | — |
| M106 | — | I97 M103 V107 | — | G96* I97* |
| | | I110 | | |
| V107 | I90 I97 | I110 | I97 S98 T99 | M106 I110 |
| | T99 | | | |
| V108 | I77 I80 | — | I80 D84 T99 | — |
| | K81 | | | |
| T109 | — | I97 | — | D95 I97 |
| I110 | — | I97 V107 I110 | — | I97 V107 I110 |
| | | L111 V114 | | M106 |
| L111 | T73 M76 | V114 | L66 M76 I77 | I110 V114 |
| | I77 I80 | | I80 F92 I97 | |
| S112 | I77 | — | I77 K81 | — |
| A113 | — | L66 D95 I97 | — | L66 D95 I97 |
| V114 | — | L66 T73 L111 | — | L66 I110 L111 |
| | | V114 E118 | | V114 |
| A115 | T73 | — | T73 I77 | — |
| Q116 | — | — | — | L66 D67 D95 |
| A117 | — | L66 D67 | — | L66 D67 |
| E118 | — | T73 E118 | T73 | V114 E118 |
| R119 | — | — | A74 | — |
| Q120 | — | D87 | — | — |
| R121 | — | L66* D67 G70* | — | R71 D72 |
| | | R71 D72* T73 | | |
| | | M76 | | |
| E124 | — | V9 S10 R68 | — | — |
| R125 | — | R71 D72 | — | — |
| R128 | — | T11 | — | — |

### REFERENCES

Avila, P., Ackroyd, A. J., and Halford, S. E. (1990). DNA binding by mutants of Tn21 resolvase with DNA recognition functions from Tn3 resolvase. J. Mol. Biol., 216, 645-655.
Blake, D. G., Boocock, M. R., Sherratt, D. J., and Stark, W. M. (1995). Cooperative binding of Tn3 resolvase monomers to a functionally asymmetric binding site. Curr. Biol., 5, 1036-1046.
Buchholz, F., and Stewart, A. F. (2001). Alteration of Cre recombinase site specificity by substrate-linked protein evolution. Nat. Biotechnol., 19, 1047-1052.
Santoro, S. W., and Schultz, P. G. (2002). Directed evolution of the site specificity of Cre recombinase. Proc. Natl. Acad. Sci. U S A, 99, 4185-4190.
Sclimenti, C. R., Thyagarajan, B., and Calos, M. P. (2001). Directed evolution of a recombinase for improved genomic integration at a native human sequence. Nucl. Acids Res., 29, 5044-5051.
Gorman, C., and Bullock, C. (2000). Site-specific gene targeting for gene expression in eukaryotes. Curr. Opin. Biotech. 11, 455-460.
Pabo, C. O., Peisach, E., and Grant, R. A. (2001). Design and selection of novel Cys₂HiS₂ zinc finger proteins. Ann. Rev. Biochem. 70, 313-340.
S. Maeser and R. Kahmann (1991). The Gin recombinase of phage Mu can catalyse site-specific recombination in plant protoplasts. Mol. Gen. Genet. 230, 170-176.
Bibikova, M., Golic, M., Golic, K.G., and Carroll, D. (2002). Targeted chromosomal cleavage and mutagenesis in Drosophila using zinc finger nucleases. Genetics 161, 1169-1175.
Shaikh and Sadowski, P, 2000. Chimeras of the Flp and Cre recombinases: tests of the mode of cleavage by Cre and Flp. J. Mol. Biol. 302, 27-48.
Nunes-Duby, S.E., et al., 1994. 1 Integrase cleaves DNA in cis. EMBO J. 13, 4421-4430.
Smith, M. C. M. and Thorpe, H. M. (2002). Diversity in the serine recombinases. Mol. Microbiol. 44, 299-307.
Schneider, F., Schwikardi, M. Muskhelishvili, G., and Droge, P. (2000). A DNA-binding domain swap converts the invertase Gin into a resolvase. J. Mol. Biol. 295, 767-775.
Elrod-Erickson, M., Rould, M. A., Nekludova, L., and Pabo, C. O. (1996) Zif268 protein-DNA complex refined at 1.6 Å. Structure 4, 1171-1180.
Kilby, N. J., Snaith, M. R., and Murray, J. A. H. (1993). Site-specific recombinases: tools for genetic engineering. Trends Genet. 9, 413-421.
Nagy, A. (2000). Cre recombinase: the universal reagent for genome tailoring. Genesis 26, 99-109.
Groth, A. C., Olivares, E. C., Thyagarajan, B., and Calos, M. P. (2000). A phage integrase directs efficient site-specific integration in human cells. Proc. Natl. Acad. Sci. USA 97, 5995-6000.
Schwikardi, M. and Droge, P. (2000). Site-specific recombination in mammalian cells catalysed by γδ resolvase mutants: implications for the topology of episomal DNA. FEBS Lett. 471, 147-150.
Choo, Y. and Isalan, M. (2000). Advances in zinc finger engineering. Curr. Opin. Truct. Biol. 10, 411-416.
Wolfe, S. A., Greisman, H. A., Ramm, E. I., and Pabo C. O. (1999). Analysis of zinc fingers optimised via phage display: evaluating the utility of a recognition code. J. Mol Biol. 285, 1917-34.
Wilmut, I., Archibald, A. L., McClenaghan, M., Simons, J. P., Whitelaw, C. B., and Clark, A. J. (1991). Production of pharmaceutical proteins in milk. Experientia 47, 905-912.
Isalan, M., Klug, A., and Choo, Y. (2001). A rapid, generally applicable method to engineer zinc fingers illustrated by targeting the HIV-1 promoter. Nat. Biotechnol. 19, 656-60.
Akopian, A., He, J., Boocock, M. R., and Stark, W. M. (2003) Chimeric site-specific recombinases with designed DNA sequence recognition. Proc. Natl. Acad. Sci. USA: in press.
Arnold, P. H., Blake, D. G., Grindley, N. D. F., Boocock, M. R., and Stark, W. M. (1999) Mutants of Tn3 resolvase which do not require accessory binding sites for recombination activity. EMBO J 18: 1407-1414.
Bednarz, A. L., Boocock, M. R., and Sherratt, D. J. (1990) Determinants of correct res site alignment in site-specific recombination by Tn3 resolvase. Genes Dev 4: 2366-2375.
Blake, D. G. (1993) Binding of Tn3 resolvase to its recombination site. Ph.D. Thesis, University of Glasgow.
Chen, Y., and Rice, P. A. (2003) New insight into site-specific recombination from FLP recombinase DNA structures. Annu. Rev. Biophys. Biomol. Struct. 32: 135-159.
Fromant, M., Blanquet, S., and Plateau, P. (1995) Direct random mutagenesis of gene-sized DNA fragments using polymerase chain reaction. Anal Biochem 224: 347-353.
Grindley, N. D. F. (1993) Analysis of a nucleoprotein complex: the synaptosome of ybresolvase. Science 262: 738-740.
Grindley, N. D. F. (1994) Resolvase-mediated site-specific recombination. In Nucleic Acids and Molecular Biology (Eckstein, F. and Lilley, D. M. J., eds) , Vol. 8, pp 236-267, Springer-Verlag, Berlin.
Grindley, N. D. F. (2002) The movement of Tn3-like elements: transposition and cointegrate resolution. In Mobile DNA II. Craig, N., Craigie, R., Gellert, M. and Lambowitz, A., (eds). Washington, DC: ASM Press, Chap. 14, pp 272-302.
Haffter, P., and Bickle, T. A. (1988) Enhancer-independent mutants of the Cin recombinase have a relaxed topological specificity. EMBO J 7: 3991-3996.
Haykinson, M. J., Johnson, L. M., Soong, J., and Johnson, R. C. (1996) The Hin dimer interface is critical for Fis-mediated activation of the catalytic steps of site-specific DNA inversion. Curr Biol 6: 163-177.
Heichman, K. A., and Johnson, R. C. (1990) The Hin invertasome: protein-mediated joining of distant recombination sites at the enhancer. Science 249: 511-517.
Hughes, R. E., Hatfull, G. F., Rice, P. A., Steitz, T. A., and Grindley, N. D. F. (1990) Cooperativity mutants of the γδ resolvase identify an essential interdimer interaction. Cell 63: 1331-1338.
Johnson, R. C. (2002) Bacterial site-specific DNA inversion systems. In Mobile DNA II. Craig, N., Craigie, R., Gellert, M. and Lambowitz, A., (eds). Washington, DC: ASM Press, Chap. 13, pp 230-271.
Kilbride, E., Boocock, M. R., and Stark, W. M. (1999) Topological selectivity of a hybrid sitespecific recombination system with elements from Tn3 res/resolvase and bacteriophage P1 loxP/Cre. J Mol Biol 289: 1219-1230.
Klippel, A., Cloppenborg, K., and Kahmann, R. (1988) Isolation and characterisation of unusual gin mutants. EMBO J 7: 3983-3989.
Leschziner, A. E., and Grindley, N. D. F. (2003) The architecture of the γδ resolvase crossover site complex revealed by using constrained DNA substrates (submitted to Mol. Cell).
Mcllwraith, M. J., Boocock, M. R., and Stark, W. M. (1997) Tn3 resolvase catalyses multiple recombination events without intermediate rejoining of DNA ends. J Mol Biol 266: 108-121.
Merickel, S. K., Haykinson, M. J., and Johnson, R. C. (1998) Communication between Hin recombinase and Fis regulatory subunits during coordinate activation of Hin-catalyzed site-specific recombination. Genes Dev 12: 2803-2816.
Murley, L. L., and Grindley, N. D. F. (1998) Architecture of the γδ resolvase synaptosome: oriented heterodimers identify interactions essential for synapsis and recombination. Cell 95: 553-562.
Nash, H. A. (1996) Site-specific recombination: integration, excision, resolution, and inversion of defined DNA segments. In Escherichia coli and Salmonella typhimurium: Cellular and Molecular Biology. Neidhart, F. C., et al., (eds). Washington, DC: American Society for Microbiology, 2nd edn., Vol. 2, pp. 2363-2376.
Pan, B., Maciejewski, M. W., Marintchev, A., and Mullen, G. P. (2001) Solution structure of the catalytic domain of γδ resolvase: implications for the mechanism of catalysis. J Mol Biol 310: 1089-1107.
Prentki, P., Binda, A., and Epstein, A. (1991) Plasmid vectors for selecting IS1-promoted deletions in cloned DNA: sequence analysis of the omega interposon. Gene 103: 17-23.
Rice, P. A., & Steitz, T. A. (1994a) Model for a DNA-mediated synaptic complex suggested by crystal packing of gamma delta resolvase subunits. EMBO J 13: 1514-1524.
Rice, P. A., & Steitz, T. A. (1994b) Refinement of γδ resolvase reveals a strikingly flexible molecule. Structure 2: 371-384.
Rowland, S-J., Stark, W. M., and Boocock, M. R. (2002) Sin recombinase from Staphylococcus aureus: synaptic complex architecture and transposon targeting. Mol Microbiol 44: 607-619.
Sanderson, M. R., Freemont, P. S., Rice, P. A., Goldman, A., Hatfull, G. F., Grindley, N. D. F., and Steitz, T. A. (1990) The crystal structure of the catalytic domain of the site-specific recombination enzyme γδ resolvase at 2.7 Å resolution. Cell 63: 1323-1329.
Sarkis, G. J., Murley, L. L., Leschziner, A. E., Boocock, M. R., Stark, W. M., and Grindley, N. D. F. (2001) A model for the γδ resolvase synaptic complex. Mol Cell 8: 623-631.
Stark, W. M., Sherratt, D. J., and Boocock, M. R. (1989) Site-specific recombination by Tn3 resolvase: topological changes in the forward and reverse reactions. Cell 58: 779-790.
Stark, W. M., and Boocock, M. R. (1994) The linkage change of a knotting reaction catalysed by Tn3 resolvase. J Mol Biol 239: 25-36.
Watson, M. A., Boocock, M. R., and Stark, W. M. (1996) Characterisation of the synaptic intermediate in site-specific recombination by Tn3 resolvase. J Mol Biol 257: 317-329.
Wells, R. G., and Grindley, N. D. F. (1984) Analysis of the γδ res site: sites required for sitespecific recombination and gene expression. J Mol Biol 179: 667-687.
Wenwieser, S. V. C. T. (2001) Subunit interactions in regulation and catalysis of site-specific recombination. Ph.D Thesis, University of Glasgow.
Yang, W., and Steitz, T. A. (1995) Crystal structure of the site-specific recombinase y6resolvase complexed with a 34 bp cleavage site. Cell 82: 193-208.
Zaccblo, M., Williams, D. M., Brown, D. M., and Gherardi, E. (1996) An approach to random mutagenesis of DNA using mixtures of triphosphate derivatives of nucleoside analogues. J Mol Biol 255: 589-603.

1. A serine recombinase comprising a catalytic domain and a DNA binding domain wherein said catalytic domain is mutated at G101 or at a position corresponding to G101 of Tn3 resolvase.

2. A serine recombinase according to claim 1 wherein the mutation is G101S.

3. A serine recombinase comprising a catalytic domain and a DNA binding domain wherein said catalytic domain is mutated at Q105 or at a position corresponding to Q105 of Tn3 resolvase.

4. A serine recombinase according to claim 3 wherein the mutation is Q105L.

5. A serine recombinase comprising a catalytic domain and a DNA binding domain wherein said catalytic domain is mutated at D102 or at a position corresponding to D102 of Tn3 resolvase, and wherein the serine recombinase is not a D102Y E124Q mutant.

6. A serine recombinase according to claim 3 wherein the mutation is selected from D102Y, D1021, D102F, D102T, D102V, D102W or D102A.

7. A serine recombinase according to claim 1 further comprising one or more additional mutations selected from the group L105Q, V107M, V107L, V107F, Q105L, A117V, R121K, E124Q, E124A, A89T, F92S, M1031 or at positions corresponding to these mutations in Tn3 resolvase.

8. A serine recombinase according to claim 1 further comprising a one or more mutations of the surface residues corresponding to a ‘2,3’ interface.

9. A serine recombinase according to claim 8 wherein the one or more mutations of the surface residues corresponding to a ‘2,3’ interface include R2A and E56K or positions corresponding to RA and E56K in Tn3 resolvase.

10. A serine recombinase according to claim 1 further comprising a one or more mutations of the surface residues corresponding to a ‘1,2’ interface.

11. A serine recombinase according to claim 10 wherein the one or more mutations of the surface residues corresponding to a ‘1,2’ interface include L66, G70, M76, M103, V107, T109, A117, R121, and E124 or positions corresponding to L66, G70, M76, M103, V107, T109, A117, R121, and E124 in Tn3 resolvase.

12. A serine recombinase according to claim 1 further comprising the mutations R2A, E56K, G101 S, D102Y, M1031 and Q105L or the positions corresponding to these mutations in Tn3 resolvase.

13. A serine recombinase according to claim 12 further comprising the mutation V107F or the position corresponding to this mutation in Tn3 resolvase.

14. A serine recombinase according to any one of the preceding claim 1 which is selected from the group consisting of Tn3 resolvase, Sin recombinase, y6 resolvase, Tn 21 resolvase, R resolvase, ISXc5 resolvase, Gin resolvase, Hin resolvase, Methanococcus jannaschii.resolvase, 15667 resolvase, ccrA1 resolvase, TN4451 resolvase, TP901-1 resolvase and OC31 resolvase.

15. A nucleic acid sequence encoding a serine recombinase according to claim 1.

16. A nucleic acid expression vector comprising a nucleic acid sequence according to claim 15.

17. A host cell comprising a nucleic acid sequence according to claim 15.

18. A hybrid recombinase comprising a catalytic domain from a serine recombinase connected by way of a linker to a heterologous DNA binding domain wherein said hybrid recombinase is capable of binding nucleic acid by way of said DNA binding domain and said catalysing recombination of said DNA.

19. A hybrid recombinase according to claim 18 wherein the heterologous DNA binding domain is the DNA binding domain of Zif268.

20. A hybrid recombinase according to claim 19 wherein the Zif268 DNA binding domain comprises a wild-type sequence starting from residue 2.

21. A hybrid recombinase according to claim 18 wherein the Zif268 DNA binding domain is mutated at one or more amino acids.

22. A hybrid recombinase according to claim 18 wherein the catalytic domain is mutated at G101 or at a position corresponding to G101 of Tn3 resolvase.

23. A hybrid recombinase according to claim 22 wherein the mutation is G101 S.

24. A hybrid recombinase according to claim 18 wherein said catalytic domain is mutated at Q105 or at a position corresponding to Q105 of Tn3 resolvase.

25. A hybrid recombinase according to claim 24 wherein the mutation is V107F.

26. A hybrid recombinase according to claim 18 wherein said catalytic domain is mutated at D102 or at a position corresponding to D102 of Tn3 resolvase.

27. A hybrid recombinase according to claim 26 wherein the mutation is selected from D102Y, D1021, D102F, D102T, D102V, D102W or D102A.

28. A hybrid recombinase according to claim 18 wherein said catalytic domain comprises one or more additional mutations selected from the group R2A, E56K, G101S, D102Y, L105Q, V107M, V107L, V107F, Q105L, A117V, R121K, E124Q, E124A, A89T, F92S, M1031 or at position corresponding to these mutations in Tn3 resolvase.

29. A hybrid recombinase according to claim 28 wherein said catalytic domain comprises the mutations R2A, E56K, G101S, D102Y, M1031 and Q105L or the positions corresponding to these mutations in Tn3 resolvase.

30. A hybrid recombinase according to claim 29 further comprising the mutation V107F or the position corresponding to this mutation in Tn3 resolvase.

31. A hybrid recombinase according to a claim 18 wherein the catalytic domain is between 125 and 146 amino acids in length.

32. A hybrid recombinase according to claim 31 wherein said catalytic domain is 125 amino acids in length.

33. A hybrid recombinase according to claim 31 wherein the catalytic domain is 146 amino acids in length.

34. A hybrid recombinase according to claim 31 wherein the catalytic domain is 140 amino acids in length.

35. A hybrid recombinase according to claim 31 wherein the catalytic domain is 144 amino acids in length.

36. A hybrid recombinase according to any claim 18 wherein the linker sequence is selected from the group consisting of TVDRSSDPTSQ, GSGGSG, GSGGSGGSG, GSGGSGGSGGSG, GGGSGGG, GGGSGGGGSGGG, TVDRSSDPTSQTS, GSGGSGTS, GSGGSGGSGTS, GSGGSGGSGGSGTS, GGGSGGGTS, GGGSGGGGSGGGTS, NRVAQQLAGKQS, SDYTQNNIHO, TVDRTS and TS.

37. A hybrid recombinase according to claim 36 wherein the linker sequence is TVDRTS.

38. A hybrid recombinase according to claim 18 wherein the catalytic domain is a Tn3 resolvase catalytic domain.

39. A hybrid recombinase comprising a Tn3 resolvase catalytic domain, which catalytic domain comprises the mutations R2A, E56K, G101S, D102Y, M1031 and Q105L and V107F, linked to a DNA binding domain via a linker comprising the sequence TS, wherein said hybrid recombinase is capable of binding nucleic acid by way of said DNA binding domain and catalysing recombination of said DNA.

40. A hybrid recombinase according to claim 39 wherein the linker comprises the sequence TVDRTS.

41. A hybrid recombinase according to claim 39 wherein the catalytic domain is amino acids 1 to 148 of a TN3 resolvase catalytic domain.

42. A hybrid recombinase according to claim 39 wherein the catalytic domain is amino acids 1 to 144 of a TN3 resolvase catalytic domain.

43. A nucleic acid sequence encoding a hybrid recombinase according to claim 18.

44. A nucleic acid expression vector comprising a nucleic acid sequence according to claim 43.

45. A host cell comprising a nucleic acid sequence according to claim 43.

46. A catalytic domain of a serine recombinase which has been mutated at G101 or at a position corresponding to G101 of Tn3 resolvase.

47. A catalytic domain according to claim 46 wherein the mutation is G101S.

48. A catalytic domain of a serine recombinase which has been mutated at Q105 or at a position corresponding to Q105 of Tn3 resolvase.

49. A catalytic domain according to claim 48 wherein the mutation is Q105L.

50. A catalytic domain of a serine recombinase which is mutated at D102 of Tn3 resolvase, and wherein the catalytic domain does not further comprise a mutation at E124Q.

51. A catalytic domain according to claim 50 wherein the mutation is selected from D102Y, D102I, D102F, D102T, D102V, D102W or d102a.

52. A catalytic domain according to claim 46 further comprising one or more additional mutations selected from the group L105Q, V107M, V107L, V107F, Q105L, A117V, R121K, E124Q, E124A, A89T, F92S, M1103I or at positions corresponding to these mutations in Tn3 resolvase.

53. A catalytic domain according to claim 46 further comprising a one or more mutations of the surface residues correesponding to a ‘2,3’ interface.

54. A catalytic domain according to claim 53 wherein the one or more mutations of the surface residues corresponding to a ‘2,3’ interface include R2A and E56K or positions corresponding to R2A and E56K in Tn3 resolvase.

55. A catalytic domain according to claim 46 further comprising a one or more mutations of the surface residues corresponding to a ‘1,2’ interface.

56. A catalytic domain according to claim 55 wherein the one or more mutations of the surface residues corresponding to a ‘1,2’ interface include L66, G70, M76, M103, V107, T109, A117, R121, and E124 or positions corresponding to L66, G70, M76, M103, V107, T109, A117, R121, and E124 in Tn3 resolvase.

57. A catalytic domain according to claim 46 further comprising the mutations R2A, E56K, G101S, D102Y, M1031 and Q105L or the positions corresponding to these mutations in Tn3 resolvase.

58. A catalytic domain according to claim 57 fuirther comprising the mutation V107F or the position corresponding to this mutation in Tn3 resolvase.

59. A catalytic domain according to claim 46 which is selected from the group consisting of Tn3 resolvase, Sin recombinase, yS resolvase, Tn 21 resolvase, 3 resolvase, ISXc5 resolvase, Gin resolvase, Hin resolvase, Methanococcus jannaschii.resolvase, 15607 resolvase, ccrA1 resolvase, TN4451 resolvase, TP901-1 resolvase and OC31 resolvase.

60. A nucleic acid sequence encoding a catalytic domain of a serine recombinase according to claim 46.

61. A nucleic acid expression vector comprising a nucleic acid sequence according to claim 60.

62. A host cell comprising a nucleic acid sequence according to claim 60 or a nucleic acid expression vector according to claim 61.

63. A method for identifying a hyperactive mutant serine recombinase capable of catalysing site-specific DNA recombination when bound to a recognition site comprising fewer nucleotides than necessary for achieving recombination with a corresponding wild-type serine recombinase, comprising the steps of
(a) mutating said wild-type serine recombinase such that the mutant recombinase comprises one or more mutations, in a catalytic domain of the recombinase, with respect to the wild-type serine recombinase; and
(b) detecting whether or not said mutant serine recombinase is capable of catalysing DNA recombination when bound to said recognition site comprising fewer nucleotides than necessary for achieving recombination with the corresponding wild-type serine recombinase

64. A method of recombining DNA comprising contacting a first DNA sequence and a second DNA sequence with a serine recombinase according to claim 1 under suitable conditions for allowing a recombination of said first and second DNA sequences.

65. A method of recombining DNA comprising contacting a first DNA sequence and a second DNA sequence with a serine recombinase according to claim 18 under suitable conditions for allowing a recombination of said first and second DNA sequences.

66. A method according to claim 64 wherein said first DNA sequence and said second DNA sequence comprise at least the 28 bp binding site I of Tn3 resolvase.

67. A kit for recombining a first DNA sequence and a second DNA sequence said kit comprising a serine recombinase according to any one of claim 1.

68. A kit for recombining a first DNA sequence and a second DNA sequence said kit comprising a hybrid recombinase according to claim 18.

69. A kit for recombining a first DNA sequence and a second DNA sequence, said kit comprising
a nucleic acid sequence according to claim 15.
